(19) United States
(12) Patent Application Publication
(10) Pub. No.: US 2010/0152081 A1
(43) Pub. Date: Jun. 17, 2010

(54) Fluorosurfactants

(76) Inventors: Wolfgang Hierse, Gross-Zimmern (DE); Nikolai (Mykola) Ignatyev, Duisburg (DE); Martin Seidel, Darmstadt (DE); Elvira Montenegro, Weinheim (DE); Peer Kirsch, Kanagawa (JP); Andreas Bathe, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/307,325

(22) PCT Filed: Jul. 2, 2007
(86) PCT No.: PCT/EP07/05841

§ 371 Date: Sep. 8, 2009

(30) Foreign Application Priority Data
Jul. 4, 2006 (DE) 10 2006 032 391.2

(51) Int. Cl. C10M 135/28; C09D 5/00; A62D 1/00; D06M 13/256; C10M 131/12; C10M 133/08; C10M 135/22; C10M 105/14; C11D 1/24; C07C 303/02; C07C 309/34; C07C 309/29

(52) U.S. Cl. 508/417; 106/287.23; 252/2; 252/8.61; 508/524; 508/562; 508/569; 508/582; 510/495; 562/76; 562/78

(57) ABSTRACT

The invention relates to compounds comprising an aryl sulfonate group, a spacer and a Y group, wherein Y represents CF₃—(CH₂)ₐ—O—, SF₅—, CF₃—(CH₂)ₐ—S—, CF₃CF₂S—, [CF₃—(CH₂)ₐ]₂N— or [CF₃—(CH₂)ₐ]NH—, a represents a whole number selected from the range of 0 to 5 or (formula I), Rf represents CF₃—(CH₂)ᵣ—, CF₃—(CH₂)ᵣ—O—, CF₃—(CH₂)ᵣ—S—, CF₃CF₂—S—, SF₅—(CH₂)ᵣ— or [CF₃—(CH₂)ᵣ]₂N—, [CF₃—(CH₂)ᵣ]NH— or (CF₃)₂N—(CH₂)ᵣ—, B represents a simple bond, O, NH, NR, CH₂, C(O)—O, C(O), S, CH₂—O, O—C(O), N—C(O)1C(O)—N, O—C(O)—N, N—C(O)—N, O—SO₂ or SO₂—O, R represents alkyl having 1 to 4 C-atoms, b represents 0 or 1 and c represents 0 or 1, q represents 0 or 1, at least one radical from b and q represents 1, and r represents 0, 1, 2, 3, 4 or 5. The invention also relates to a method for the production of said compounds and to the uses of said surface-active compounds.

The present invention relates to compounds containing an arylsulfonate group, a spacer and a group Y, where Y stands for CF₃—(CH₂)ₐ—O—, SF₅—, CF₃—(CH₂)ₐS—, CF₃CF₂S—, [CF₃—(CH₂)ₐ]₂N— or [CF₃—(CH₂)ₐ]NH—, where a stands for an integer selected from the range from 0 to 5, or

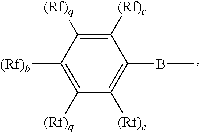

where

Rf stands for CF₃—(CH₂)ᵣ, CF₃—(CH₂)ᵣO—, CF₃—(CH₂)ᵣ—S—, CF₃CF₂—S—, SF₅—(CH₂)ᵣ— or [CF₃—(CH₂)ᵣ]₂N—, [CF₃—(CH₂)ᵣ]NH— or (CF₃)₂N—(CH₂)ᵣ—, B stands for a single bond, O, NH, NR, CH₂, C(O)—O, C(O), S, CH₂—O, O—C(O), N—C(O), C(O)—N, O—C(O)—N, N—C(O)—N, O—SO₂ or SO₂—O,

R stands for alkyl having 1 to 4 C atoms,

b stands for 0 or 1 and c stands for 0 or 1,

q stands for 0 or 1, where at least one radical from b and q stands for 1, and

r stands for 0, 1, 2, 3, 4 or 5, to processes for the preparation of these compounds, and to uses of these surface-active compounds.

Fluorosurfactants have an outstanding ability to reduce surface energy, which is utilised, for example, in the hydrophobicisation of surfaces, such as textile impregnation, hydrophobicisation of glass, or de-icing of aircraft wings.

In general, however, fluorosurfactants contain perfluoroalkyl substituents, which are degraded in the environment by biological and other oxidation processes to give perfluoroalkanecarboxylic acids and -sulfonic acids. These are regarded as persistent and are in some cases suspected of causing health damage (G. L. Kennedy, Jr., J. L. Butenhoff, G. W. Olsen, J. C. O'Connor, A. M. Seacat, R. G. Perkins, L. B. Biegel, S. R. Murphy, D. G. Farrar, Critical Reviews in Toxicology 2004, 34, 351-384). In addition, relatively long-chain perfluoroalkanecarboxylic acids and -sulfonic acids accumulate in the food chain.

There is therefore a demand for surface-active substances which have a property profile comparable to the classical fluorosurfactants and which can preferably be degraded oxidatively or reductively. Particularly advantageous compounds here are those which do not leave behind any persistent organofluorine degradation products on oxidative or reductive degradation.

A known perfluorinated arylsulfonate is the sodium salt of 4-(heptadeca-fluorooctyl)benzenesulfonic acid (CAN 118:126988).

The Omnova company markets polymers whose side chains contain terminal CF₃ or C₂F₅ groups. International patent application WO 03/010128 describes perfluoroalkyl-substituted amines, acids, amino acids and thio-ether acids which contain a C₃₋₂₀-perfluoroalkyl group.

JP-A-2001/133984 discloses surface-active compounds containing per-fluoroalkoxy chains which are suitable for use in antireflection coatings. JP-A-09/111286 discloses the use of perfluoropolyether surfactants in emulsions.

However, these known fluorosurfactants ultimately result in the formation of persistent perfluoroalkanesulfonic acids and -carboxylic acids on degradation. Even the substitutes containing a terminal CF₃ group which have been introduced as being more ecologically friendly can be degraded to give persistent trifluoroacetic acid.

The earlier German patent application DE 102005000858 describes compounds which carry at least one terminal pentafluorosulfuranyl group or at least one terminal trifluoromethoxy group and contain a polar end group, are surface-active and are highly suitable as surfactants.

Linear arylsulfonates with no fluorination, for example sodium nonyl-benzenesulfonate, are amongst the most-used surfactants since they are biodegradable. They have good chemical and thermal stability and can be used in spray-dried formulations. Owing to their inexpensive preparation, the linear arylsulfonates are employed as domestic detergents, as washing agents and in industrial products.

This class of arylsulfonates containing OCF₃ or SF₅ groups as modification was not described in DE 102005000858.

There continues to be a demand for further, preferably degradable substitutes for perfluorinated surfactants.

It has now been found that the compounds according to the invention containing at least one group Y selected from the group CF₃—(CH₂)ₐ—O—, SF₅—, CF₃—(CH₂)ₐ—S—, CF₃CF₂S—, [CF₃—(CH₂)ₐ]₂N— or [CF₃—(CH₂)ₐ]NH—, where a stands for an integer selected from the range from 0 to 5, or

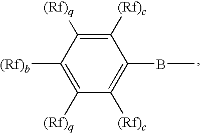

where

Rf stands for CF₃—(CH₂)ᵣ, CF₃—(CH₂)ᵣ—O—, CF₃—(CH₂)ᵣ—S—, CF₃CF₂—S—, SF₅—(CH₂)ᵣ— or [CF₃—(CH₂)ᵣ]₂N—, [CF₃—(CH₂)ᵣ]NH— or (CF₃)₂N—(CH₂)ᵣ—, B stands for a single bond, O, NH, NR, CH₂, C(O)—O, C(O), S, CH₂—O, O—C(O), N—C(O), C(O)—N, O—C(O)—N, N—C(O)—N, O—SO₂ or SO₂—O,

R stands for alkyl having 1 to 4 C atoms,

b stands for 0 or 1 and c stands for 0 or 1,

q stands for 0 or 1, where at least one radical from b and q stands for 1, and

r stands for 0, 1, 2, 3, 4 or 5, which is preferably arranged in the terminal position, are surface-active and are highly suitable as surfactants.

The invention therefore relates firstly to compounds containing an aryl-sulfonate group, a spacer and a group Y, where Y stands for CF₃—(CH₂)ₐ—O—, SF₅—, CF₃—(CH₂)ₐ—S—, CF₃CF₂S—, [CF₃—(CH₂)ₐ]₂N— or [CF₃—(CH₂)ₐ]NH—, where a stands for an integer selected from the range from 0 to 5, or

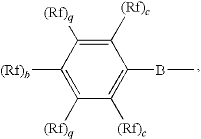

where

Rf stands for CF₃—(CH₂)ᵣ—, CF₃—(CH₂)ᵣ—O—, CF₃—(CH₂)ᵣ—S—, CF₃CF₂—S—, SF₅—(CH₂)ᵣ— or [CF₃—(CH₂)ᵣ]₂N—, [CF₃—(CH₂)ᵣ]NH— or (CF₃)₂N—(CH₂)ᵣ—,

B stands for a single bond, O, NH, NR, CH₂, C(O)—O, C(O), S, CH₂—O, O—C(O), N—C(O), C(O)—N, O—C(O)—N, N—C(O)—N, O—SO₂ or SO₂—O,

R stands for alkyl having 1 to 4 C atoms,

b stands for 0 or 1 and c stands for 0 or 1,

q stands for 0 or 1, where at least one radical from b and q stands for 1, and

r stands for 0, 1, 2, 3, 4 or 5.

The compounds according to the invention preferably contain no further fluorinated groups besides the fluorinated end groups mentioned.

The group Y in the surface-active compounds is preferably bonded to a saturated or unsaturated, branched or unbranched hydrocarbon unit, the spacer, where the hydrocarbon unit in the chain or in a branch may optionally be provided with one or more heteroatoms. The hydrocarbon units can be aliphatic or aromatic units.

In a variant of the invention, the group Y occurs a number of times in the surface-active compound, and the surface-active compound is preferably an oligomer or polymer.

In another, likewise preferred variant of the invention, the group Y occurs only once, twice or three times in the surface-active compound, where compounds in which the group occurs only once are particularly preferred.

The compounds according to the invention are preferably low-molecular-weight compounds of the formulae IA to IB

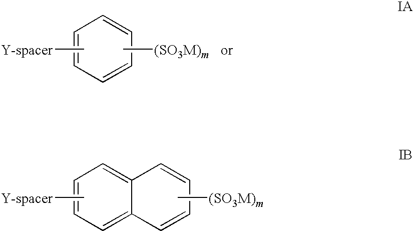

where Y stands for CF₃—(CH₂)ₐ—O—, SF₅—, CF₃—(CH₂)ₐ—S—, CF₃CF₂S—, [CF₃—(C₂)ₐ]₂N— or [CF₃—(CH₂)ₐ]NH—, where a stands for an integer selected from the range from 0 to 5, or

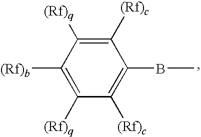

where

Rf stands for CF₃—(CH₂)ᵣ—, CF₃—(CH₂)ᵣ—O—, CF₃—(CH₂)ᵣ—S—, CF₃CF₂—S—, SF₅—(CH₂)ᵣ— or [CF₃—(CH₂)ᵣ]₂N—, [CF₃—(CH₂)ᵣ]NH— or (CF₃)₂N—(CH₂)ᵣ—,

B stands for a single bond, O, NH, NR, CH₂, C(O)—O, C(O), S, CH₂—O, O—C(O), N—C(O), C(O)—N, O—C(O)—N, N—C(O)—N, O—SO₂ or SO₂—O,

R stands for alkyl having 1 to 4 C atoms,

b stands for 0 or 1 and c stands for 0 or 1,

q stands for 0 or 1, where at least one radical from b and q stands for 1, and

r stands for 0, 1, 2, 3, 4 or 5,

spacer denotes a saturated or unsaturated, branched or unbranched hydrocarbon unit, where the hydrocarbon unit in the chain or in a branch may optionally be provided with one or more heteroatoms,

M denotes a metal cation, and

m denotes 1, 2 or 3.

In accordance with the invention, compounds of the formulae IA and/or IB are particularly preferred.

In the formulae IA and IB, M denotes a metal cation, in particular selected from an alkali metal cation, an alkaline-earth metal cation or an ammonium ion. The lithium, sodium or potassium cation or NH₄⁺ is preferably used for M.

In the formulae IA and IB, m indicates the number of sulfonyl radicals present, where m is preferably 1 or 2, very particularly preferably 1.

For m=1, the position of the sulfonyl radical for the formula IA is possible in the ortho-, meta- or para-position. The para-position is preferred.

For m=1, the position of the sulfonyl radical for the formula IB is preferably on carbon atom C-4, C-5 or C-6, particularly preferably on C-5 or C-6, which is illustrated by the following sub-formulae:

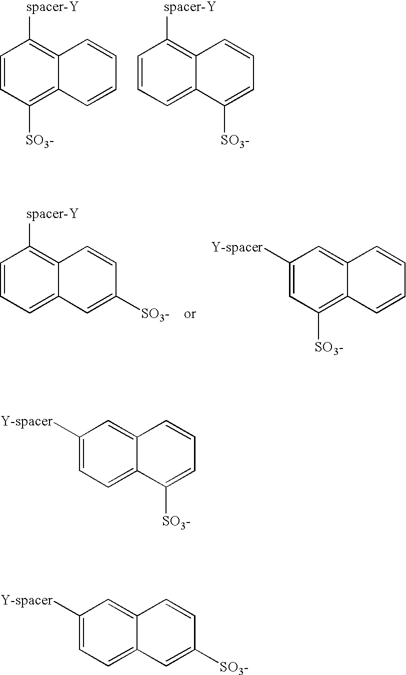

The position of the spacer which carries the group Y is preferably on C-1 or C-2, particularly preferably on C-1.

Heteroatoms in the hydrocarbon unit of the spacer can be, for example, O, S or NH, preferably O or S, very particularly preferably O.

In a further preferred embodiment of the compounds according to the invention, the spacer can be described in formula terms by

(CH₂)ₙ—,

—(CH₂)—CH(Hal)-(CH₂)₍ₙ₋₁₎—,

—CH═CH—(CH₂)₍ₙ₋₁₎—,

—(CH₂)ₙ—O—,

—(CH₂)ₙ—O—(CH₂)ₚ— or

—CH₂CH═CH—(CH₂)₍ₙ₋₁₎—,

—(CH₂)ₙ₋₁—Ar—(CH₂)₍ₙ₋₁₎—,

—(CH₂)ₙ₋₁—C≡C—(CH₂)ₙ—,

—(CH₂)ₙ-Q-(CH₂)ₙ—,

where

Ar stands for aryl,

Q stands for O, S or N,

n and n′ stand for an integer from the range 1 to 30,

Hal denotes Cl, Br or I, and

p stands for an integer from the range 1 to 4.

n and/or n′ preferably stand for an integer from the range from 4 to 24 and particularly preferably for an integer from the range 6 to 18. It is in turn preferred in a variant of the invention for n to be an even number.

In a variant of the invention, Hal preferably stands for Cl or Br, very particularly preferably for Br.

In a variant of the invention, p preferably stands for 2 or 3, very particularly preferably for 3.

In a variant of the invention, the spacers

—(CH₂)—CH(Hal)-(CH₂)₍ₙ₋₁₎—,

—CH═CH—(CH₂)₍ₙ₋₁₎—,

are preferred for Y═SF₅.

In a variant of the invention, the spacers

—(CH₂)ₙ—,

—CH═CH—(CH₂)₍ₙ₋₁₎—,

—(CH₂)ₙ—O—,

—(CH₂)ₙ—O—(CH₂)ₚ— or

—CH₂CH═CH—(CH₂)₍ₙ₋₁₎—,

are preferred for Y═CF₃—(CH₂)ₐ—O—, CF₃—(CH₂)ₐ—S—, CF₃CF₂S—, [CF₃—(CF₂)ₐ]₂N— or [CF₃—(CH₂)ₐ]NH—, where a stands for an integer selected from the range from 0 to 5, or

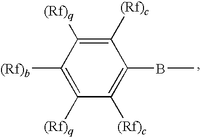

where

Rf stands for CF₃—(CH₂)ᵣ—, CF₃—(CH₂)ᵣ—O—, CF₃—(CH₂)ᵣ—S—, CF₃CF₂—S—, SF₅—(CH₂)ᵣ— or [CF₃—(CH₂)ᵣ]₂N—, [CF₃—(CH₂)ᵣ]NH— or (CF₃)₂N—(CH₂)ᵣ—,

B stands for a single bond, O, NH, NR, CH₂, C(O)—O, C(O), S, CH₂—O, O—C(O), N—C(O), C(O)—N, O—C(O)—N, N—C(O)—N, O—SO₂ or SO₂—O,

R stands for alkyl having 1 to 4 C atoms,

b stands for 0 or 1 and c stands for 0 or 1,

q stands for 0 or 1, where at least one radical from b and q stands for 1, and

r stands for 0, 1, 2, 3, 4 or 5.

In the group Y, a preferably stands for 0, 1 or 2, particularly preferably for 0 or 2, very particularly preferably for 0.

Of the fluorine groups as aryl substituents, which are also abbreviated to Rf below, preference is given to those in which r stands for 0, 1 or 2, where r preferably stands for 0. Particular preference is given in accordance with the invention to the groups Rf═CF₃—, CF₃—S—, CF₃CF₂—S—, SF₅— or (CF₃)₂N—.

In a preferred variant of the invention, the group Y, as defined above, consists of CF₃—O—, CF₃—S—, CF₃CF₂—S—, (CF₃)₂N—, CF₃NH— or

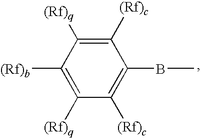

where

Rf stands for CF₃—(CH₂)ᵣ—, CF₃—(CH₂)ᵣ—O—, CF₃—(CH₂)ᵣ—S—, CF₃CF₂—S—, SF₅—(CH₂)ᵣ— or [CF₃—(CH₂)ᵣ]₂N—, [CF₃—(CH₂)ᵣ]NH— or (CF₃)₂N—(CH₂)ᵣ—,

B stands for a single bond, O, NH, NR, CH₂, C(O)—O, C(O), S, CH₂—O, O—C(O), N—C(O), C(O)—N, O—C(O)—N, N—C(O)—N, O—SO₂ or SO₂—O,

R stands for alkyl having 1 to 4 C atoms,

b stands for 0 or 1 and c stands for 0 or 1,

q stands for 0 or 1, where at least one radical from b and q stands for 1, and

r stands for 0.

Rf preferably stands for CF₃—(CH₂)ᵣ—, CF₃—(CH₂)ᵣ—O—, CF₃—(CH₂)ᵣ—S or [CF₃—(CH₂)ᵣ]₂N—. A preferred variant of the invention encompasses fluorine groups, also abbreviated to Rf below, in which r stands for 0, 1, 2 or 3, in particular for 0, 1 or 2, where r preferably stands for 0.

In a particularly preferred embodiment of the present invention, Rf stands for CF₃—, CF₃—O—, CF₃—CH₂—CH₂—O—, CF₃—S—, CF₃CF₂—S—, SF₅—, CF₃—CH₂—CH₂—S—, (CF₃)₂—N— and (CF₃—CH₂—CH₂)₂—N—, in particular for CF₃—, CF₃—O—, CF₃—S— and (CF₃)₂—N—.

A further preferred variant of the invention encompasses the groups Rf which are equal to CF₃—, CF₃—S—, CF₃CF₂—S—, SF₅— or (CF₃)₂N—.

Particularly preferred groups B are O, S, CH₂O, CH₂, C(O) and OC(O). In particular, B equal to O and OC(O) are preferred.

A particularly preferred variant of the invention encompasses the groups Y which are equal to CF₃—Ar—O, CF₃—O—Ar—O, CF₃—CH₂—CH₂—O—Ar—O, CF₃—S—Ar—O, CF₃CF₂—S—Ar—O, SF₅—Ar—O, CF₃—CH₂—CH₂—S—Ar—O, (CF₃)₂—N—Ar—O, (CF₃—CH₂—CH₂)₂—N—Ar—O, CF₃—Ar—OC(O), CF₃—O—Ar—OC(O), CF₃—CH₂—CH₂—O—Ar—OC(O), CF₃—S—Ar—OC(O), CF₃CF₂—S—Ar—OC(O), SF₅—Ar—OC(O), CF₃—CH₂—CH₂—S—Ar—OC(O), (CF₃)₂—N—Ar—OC(O) and (CF₃—CH₂—CH₂)₂—N—Ar—OC(O), in particular equal to CF₃—Ar—O, CF₃—O—Ar—O, CF₃—S—Ar—O, (CF₃)₂—N—Ar—O, CF₃—Ar—OC(O), CF₃—O—Ar—OC(O), CF₃—S—Ar—OC(O) and (CF₃)₂—N—Ar—OC(O).

A particularly preferred variant of the invention encompasses Y equal to CF₃—Ar—O and CF₃—Ar—OC(O).

In a variant of the present invention, it is preferred for q to stand for 0 and for at least one c and/or b each to stand for 1. It is particularly preferred for all c and b to stand for 1, i.e. the aromatic rings are substituted by fluorine groups in the o,p,o-position.

In a further variant of the invention, it is preferred for all q and b each to stand for 0 and for at least one c to stand for 1. It is particularly preferred for both c to stand for 1, i.e. the aromatic rings are substituted by fluorine groups in the o,o-position.

In a further variant of the invention, it is preferred for all c and q each to stand for 0 and for b to stand for 1, i.e. the aromatic rings are substituted by fluorine groups in the p-position.

Particular preference is given to the use of compounds which have a combination of the variables in their preferred or particularly preferred ranges.

Further preferred combinations are disclosed in the claims.

Advantages of the compounds according to the invention or the compositions or agents according to the invention may be, in particular:
a surface activity which may be equal or superior to the conventional hydrocarbon surfactants with respect to efficiency and/or effectiveness and/or
biological and/or abiotic degradability of the substances without the formation of persistent, perfluorinated degradation products and/or
good processability in formulations and/or
storage stability.

The particularly preferred compounds here include the following compounds:

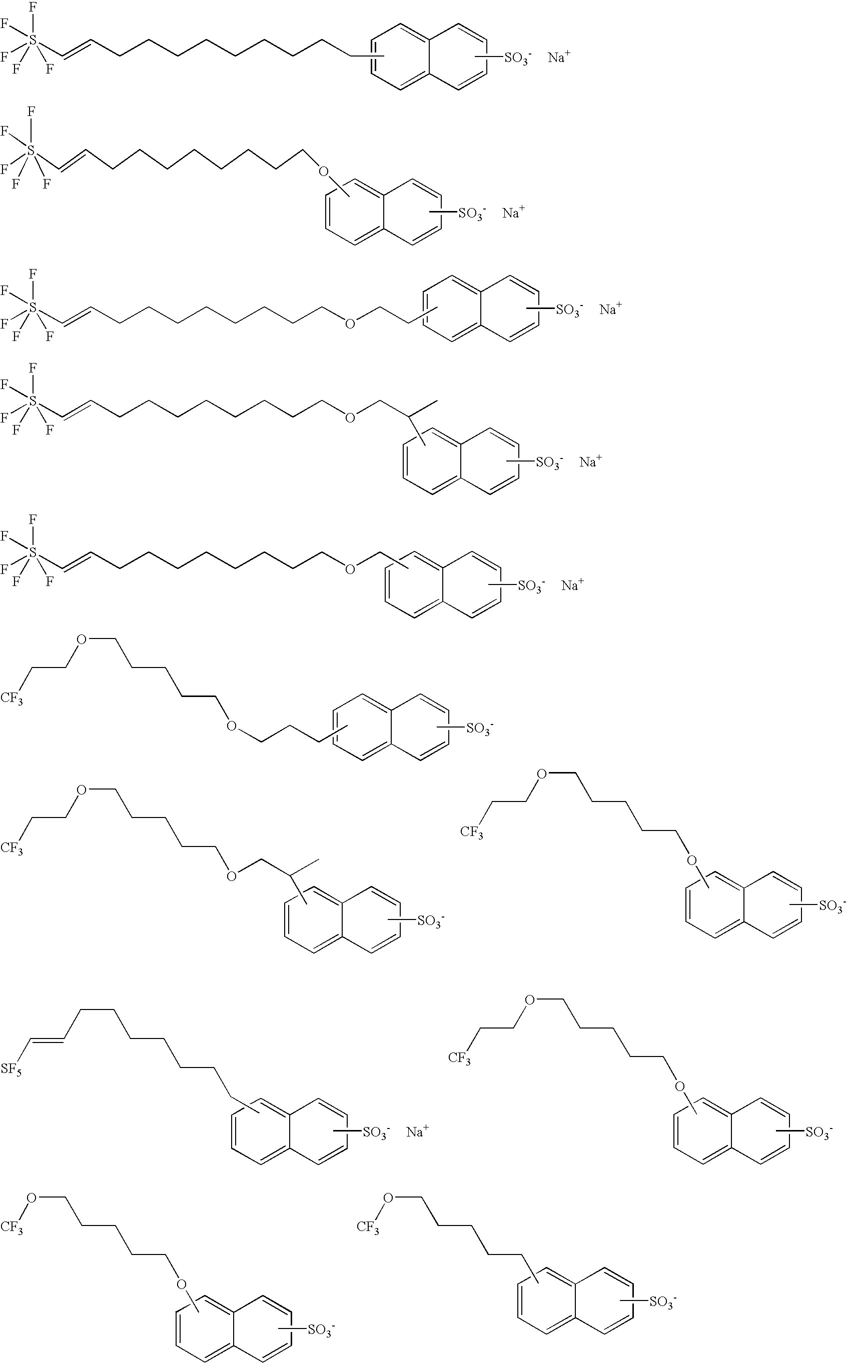

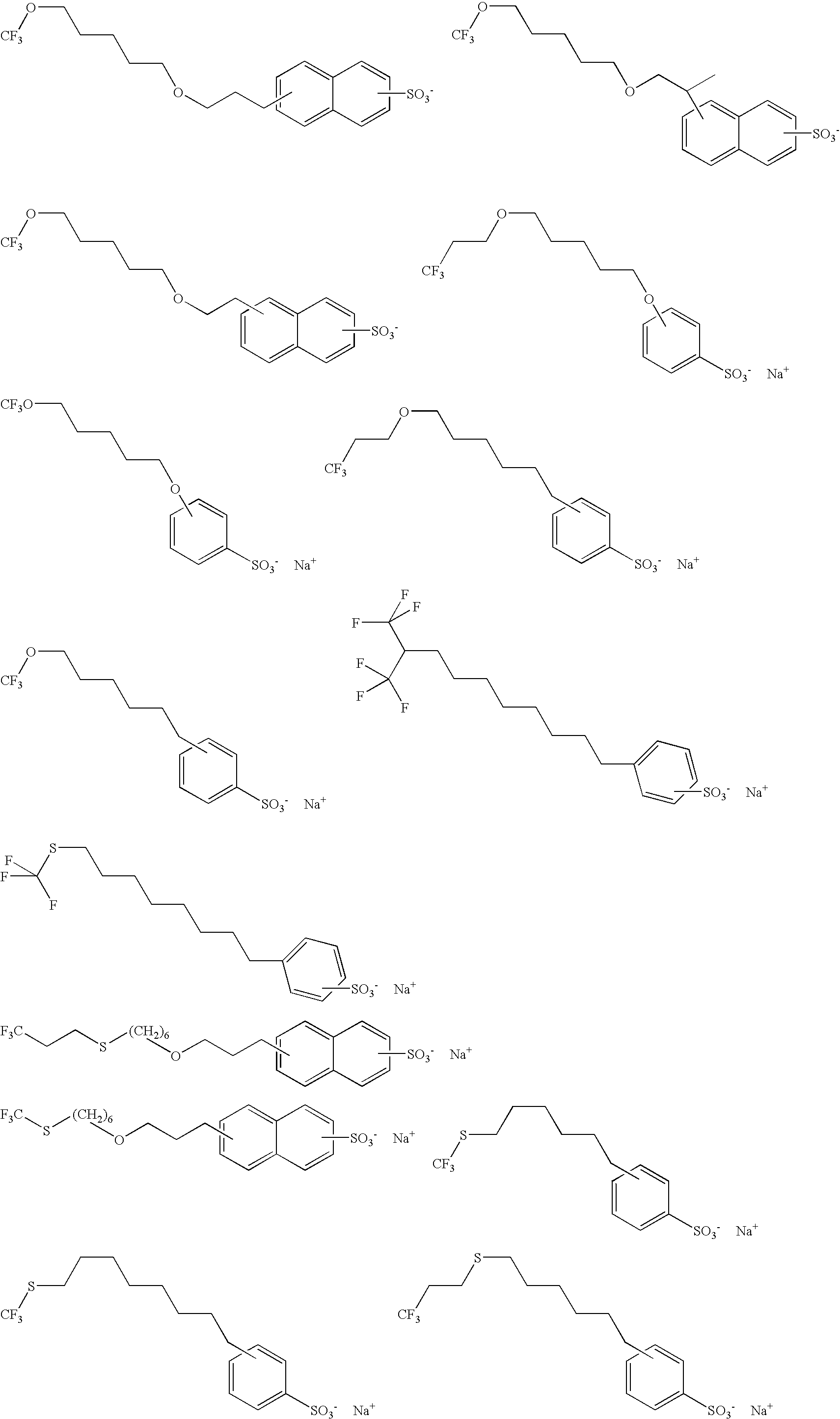

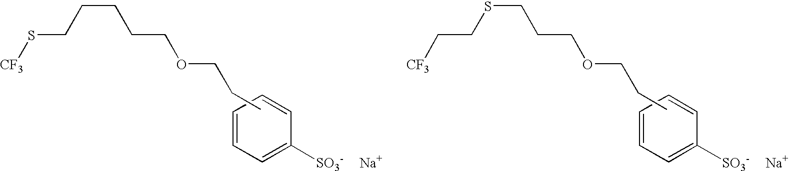

The compounds which can be used in accordance with the invention as surfactants are particularly suitable for use as hydrophobicising agents or oleophobicising agents.

Areas of use are, for example, the surface modification of textiles, paper, glass, porous building materials or adsorbents. In paints, coatings, inks, photographic coatings (for photographic plates, films and papers), special coatings for semiconductor photolithography (photoresists, top antireflective coatings, bottom antireflective coatings) or other preparations for surface coating, the compounds according to the invention and the compounds to be employed in accordance with the invention can advantageously be employed with one or more of the following functions: antifogging agent, dispersant, emulsion stabiliser, antifoam, deaerating agent, antistatic, flame retardant, gloss enhancer, lubricant, pigment- or filler-compatibility enhancer, scratch-resistance enhancer, substrate adhesion enhancer, surface-adhesion reducer, skin preventer, hydrophobicising agent, oleophobicising agent, UV stabiliser, wetting agent, flow-control agent, viscosity reducer, migration inhibitor, drying accelerator. In printing inks, the compounds according to the invention and the compounds to be employed in accordance with the invention can likewise advantageously be employed and have one or more of the following functions: antifoam, deaerating agent, friction-control agent, wetting agent, flow-control agent, pigment-compatibility enhancer, print-resolution enhancer, drying accelerator.

The present invention therefore furthermore relates to the use of the compounds according to the invention or the compounds to be employed in accordance with the invention as additives in preparations for surface coating, such as printing inks, paints, coatings, photographic coatings, special coatings for semiconductor photolithography, such as photoresists, top antireflective coatings, bottom antireflective coatings, or in additive preparations for addition to corresponding preparations.

A further use according to the invention of compounds according to the invention or compounds to be employed in accordance with the invention is the use as interface promoter or emulsifier. These properties can advantageously be utilised, in particular, for the preparation of fluoropolymers by means of emulsion polymerisation,

Compounds according to the invention and compounds to be employed in accordance with the invention can be employed as foam stabiliser, in particular in preparations which are known as “fire-extinguishing foams”. The invention therefore furthermore relates to the use of compounds according to the invention or compounds to be employed in accordance with the invention as foam stabiliser and/or for supporting film formation, in particular in aqueous film-forming fire-extinguishing foams, both synthetic and also protein-based, and also for alcohol-resistant formulations (AFFF and AFFF-AR, FP, FFFP and FFFP-AR fire-extinguishing foams).

Compounds according to the invention and compounds to be employed in accordance with the invention can also be used as antistatics. The antistatic action is of particular importance in the treatment of textiles, in particular clothing, carpets and carpeting, upholstery in furniture and automobiles, non-woven textile materials, leather goods, papers and cardboard articles, wood and wood-based materials, mineral substrates, such as stone, cement, concrete, plaster, ceramics (glazed and unglazed tiles, earthen-ware, porcelain) and glasses, and for plastics and metallic substrates. The present application relates to the corresponding use.

For metallic substrates, the present invention additionally also relates to the use of compounds according to the invention in anticorrosion agents.

The present invention furthermore also relates to the use thereof as mould-release agents in plastics processing.

In general, compounds according to the invention and compounds to be employed in accordance with the invention are suitable as protection agents against spots and soiling, stain releases, antifogging agents, lubricants, and as abrasion-resistance and mechanical wear-resistance enhancers.

Compounds according to the invention and compounds to be employed in accordance with the invention can advantageously be employed as additives in cleaning compositions and spot removers for textiles (in particular clothing, carpets and carpeting, upholstery in furniture and automobiles) and hard surfaces (in particular kitchen surfaces, sanitary installations, tiles, glass) and in polishes and waxes (in particular for furniture, flooring and automobiles) with one or more of the following functions: wetting agent, flow-control agent, hydrophobicising agent, oleophobicising agent, protection agent against spots and soiling, lubricant, antifoam, deaerating agent, drying accelerator. In the case of cleaning compositions and spot removers, the use as detergent or dirt emulsifier and dispersant is additionally also an advantageous embodiment of the present invention. The invention therefore furthermore relates to the use of compounds according to the invention or compounds to be employed in accordance with the invention in cleaning compositions and spot removers or as wetting agent, flow-control agent, hydrophobicising agent, oleophobicising agent, protection agent against spots and soiling, lubricant, antifoam, deaerating agent or drying accelerator.

The compounds according to the invention and compounds to be employed in accordance with the invention can also advantageously be used as additives in polymeric materials (plastics) with one or more of the following functions: lubricant, internal-friction reducer, UV stabiliser, hydrophobicising agent, oleophobicising agent, protection agent against spots and soiling, coupling agent for fillers, flame retardant, migration inhibitor (in particular against migration of plasticisers), antifogging agent.

On use as additives in liquid media for cleaning, etching, reactive modification and/or substance deposition on metal surfaces (in particular also electroplating and anodisation) or semiconductor surfaces (in particular for semiconductor photolithography), compounds according to the invention and compounds to be employed in accordance with the invention act as developer, stripper, edge bead remover, etching and cleaning composition, as wetting agent and/or deposited film quality enhancer. In the case of electroplating processes (in particular chrome plating), the present invention additionally also relates to the function as fume inhibitor with or without foam action.

In addition, the compounds which can be used in accordance with the invention as surfactants are suitable for washing and cleaning applications, in particular of textiles. Cleaning and polishing of hard surfaces is also a possible area of application for the compounds which can be used in accordance with the invention as surfactants. Furthermore, the compounds which can be used in accordance with the invention as surfactants can advantageously be employed in cosmetic products, such as, for example, foam baths and hair shampoos, or as emulsifiers in creams and lotions. The compounds according to the invention and the compounds to be employed in accordance with the invention can likewise advantageously be employed as additives in hair- and bodycare products (for example hair rinses and hair conditioners), with one or more of the following functions: wetting agent, foaming agent, lubricant, antistatic, skin-grease resistance enhancer.

Compounds according to the invention and compounds to be employed in accordance with the invention act as additives in herbicides, pesticides and fungicides, with one or more of the following functions: substrate wetting agent, adjuvant, foam inhibitor, dispersant, emulsion stabiliser.

Compounds according to the invention and compounds to be employed in accordance with the invention can likewise beneficially be employed as additives in adhesives, with one or more of the following functions: wetting agent, penetration agent, substrate adhesion enhancer, antifoam.

Compounds according to the invention and compounds to be employed in accordance with the invention can also serve as additives in lubricants and hydraulic fluids, with one or more of the following functions: wetting agent, corrosion inhibitor. In the case of lubricants, the use as dispersant (in particular for fluoropolymer particles) is additionally also an essential aspect.

On use as additives in putty and filling compositions, compounds according to the invention and compounds to be employed in accordance with the invention can act with one or more of the following functions: hydrophobicising agent, oleophobicising agent, protection agent against soiling, weathering-resistance enhancer, UV stabiliser, silicone bleeding inhibitor.

A further area of application for the compounds which can be used in accordance with the invention as surfactants is flotation, i.e. the recovery and separation of ores and minerals from dead rock. To this end, they are employed as additives in preparations for ore processing, in particular flotation and leaching solutions, with one or more of the following functions: wetting agent, foaming agent, foam inhibitor. A related use is, also as additives in agents for the stimulation of oil wells, with one or more of the following functions: wetting agent, foaming agent, emulsifier.

In addition, they can be employed as additives in de-icing agents or icing inhibitors.

In addition, preferred compounds which can be used in accordance with the invention as surfactants can also be employed as emulsifiers or dispersion assistants in foods. Further fields of application are in metal treatment, as leather auxiliaries, construction chemistry and in crop protection.

Surfactants according to the invention are furthermore also suitable as antimicrobial active compound, in particular as reagents for antimicrobial surface modification.

The present invention relates to all uses mentioned here of compounds to be employed in accordance with the invention. The respective use of surfactants for the said purposes is known to the person skilled in the art, and consequently the use of the compounds to be employed in accordance with the invention presents no problems.

For the application, the compounds according to the invention are usually introduced into appropriately formulated preparations. The present invention likewise relates to corresponding compositions comprising at least one compound according to the invention. Such compositions preferably comprise a vehicle which is suitable for the particular application and optionally further specific active compounds and/or optionally assistants.

Preferred compositions here are paint and coating preparations, fire-extinguishing compositions, lubricants, washing and cleaning compositions, de-icers or hydrophobicising agents for textile finishing or glass treatment. In a preferred variant of the invention, the compositions are hydrophobicising agents for finishing textiles and carpets.

For the hydrophobic finishing of textiles, hydrophobicising agents based on polysiloxanes, fluorinated hydrocarbons or mixtures of aluminium or zirconium salts with paraffins are generally employed (cf. in this respect “Handbuch der Textilhilfsmittel” [Handbook of Textile Assistants], A. Chwala, V. Anger, Verlag Chemie, New York 1977, Chapter 3.24 “Phobiermittel” [Proofing Agents], pages 735 ff.). The hydrophobic finishing of textiles, in particular in the case of weather-protection clothing, serves to make these either water-resistant or waterproof. The hydrophobicising agent is applied to the fibres of the textiles, where it aligns itself in such a way that the hydrophobic parts of the molecules are perpendicular to the fibre surface. In this way, the tendency of water to spread over the entire surface is greatly reduced. The water adopts a spherical shape owing to cohesion forces and runs off the textile surface in the form of beads.

Further areas of application for compositions according to the invention are paint and coating preparations, fire-extinguishing compositions (powders and foams), lubricants, washing and cleaning compositions and de-icers.

The compositions can be prepared by methods known per se; for example by mixing the compounds according to the invention with a vehicle which is suitable for the particular application and optionally further specific active compounds and optionally assistants. The compounds to be used in accordance with the invention can be prepared by methods known per se to the person skilled in the art from the literature.

The compounds according to the invention can be prepared by methods known per se to the person skilled in the art from the literature. Examples are described below.

The invention therefore furthermore relates to a process for the preparation of the compounds according to the invention, characterised in that a compound of the formula II

Y-spacer-Z II,

where Y and -spacer- can have one of the meanings indicated above, and Z denotes OH, Br, Cl or vinyl,

is reacted with the corresponding aromatic compound selected from the group of benzene and naphthalene, and a sulfonation and salt formation are subsequently carried out.

The bonding of the compounds of the formula II, as defined above, is preferably carried out via a Friedel-Crafts alkylation. The reaction conditions and reaction procedures for Friedel-Crafts alkylations are known to the person skilled in the art and are described in detail in the standard literature. The reaction of the compounds of the formula II with the aromatic compound is carried out in the presence of the Lewis acid aluminium trichloride. Both alcohols modified by Y and also chlorides or bromides, or compounds of the formula II containing a terminal double bond can be employed here. The synthesis of these compounds is described below.

The sulfonation by Friedel-Crafts alkylation is likewise a standard method and can be carried out, for example, by reaction with sulfur trioxide. The subsequent salt formation in alkali metal hydroxide/water gives the corresponding salts.

As an alternative to a Friedel-Crafts alkylation, a Grignard reaction is also possible. This reaction is also known as a standard reaction in expert circles.

The synthesis of the compounds of the formula II

Y-spacer-Z II,

where

the spacer is described in formula terms by

—(CH₂)ₙ—,

—(CH₂)—CH(Hal)-(CH₂)₍ₙ₋₁₎—,

—CH═CH—(CH₂)₍ₙ₋₁₎—,

—(CH₂)ₙ—O—,

—(CH₂)ₙ—O—(CH₂)ₚ— or

—CH₂CH═CH—(CH₂)₍ₙ₋₁₎—,

—(CH₂)ₙ₋₁—Ar—(CH₂)_{(n′-1)}—,

—(CH₂)ₙ₋₁—C≡C—(CH₂)ₙ—,

—(CH₂)ₙ-Q-(CF₂)_{n′}—,

where

Ar stands for aryl,

Q stands for O, S or N,

n and n′ stand for an integer from the range 1 to 30,

Hal denotes Cl, Br or I, and

p stands for an integer from the range 1 to 4, and

Y stands for CF₃—(CH₂)ₐ—O—, SF₅—, CF₃—(CH₂)ₐ—S—, CF₃CF₂S—, [CF₃—(CH₂)ₐ]₂N— or [CF₃—(CH₂)ₐ]NH—, where a stands for an integer selected from the range from 0 to 5, or

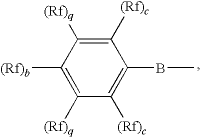

where

Rf stands for CF₃—(CH₂)ᵣ—, CF₃—(CH₂)ᵣ—O—, CF₃—(CH₂)ᵣ—S—, CF₃CF₂—S—, SF₅—(CH₂)ᵣ— or [CF₃—(CH₂)ᵣ]₂N—, [CF₃—(CH₂)ᵣ]NH— or (CF₃)₂N—(CH₂)ᵣ—,

B stands for a single bond, O, NH, NR, CH₂, C(O)—O, C(O), S, CH₂—O, O—C(O), N—C(O), C(O)—N, O—C(O)—N, N—C(O)—N, O—SO₂ or SO₂—O,

R stands for alkyl having 1 to 4 C atoms,

b stands for 0 or 1 and c stands for 0 or 1,

q stands for 0 or 1, where at least one radical from b and q stands for 1, and

r stands for 0, 1, 2, 3, 4 or 5, and

Z═OH, Cl, Br or vinyl,

is now described in detail for each group Y.

The groups Y can generally be introduced in accordance with the following schemes:

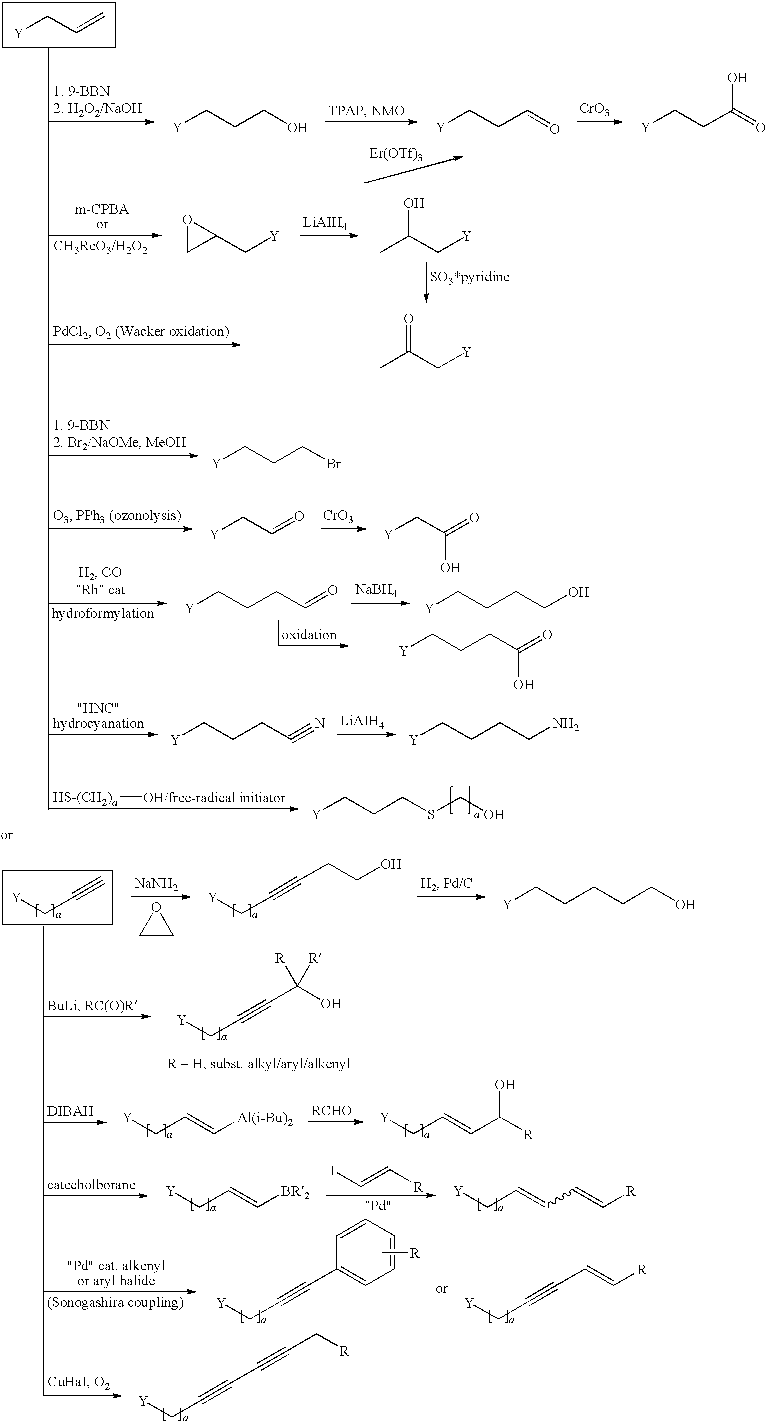

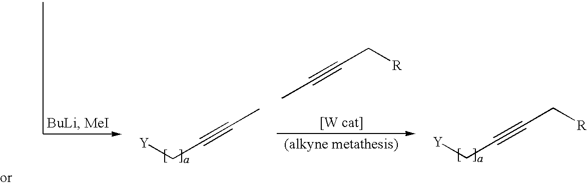

Chain extension by cross-metathesis:

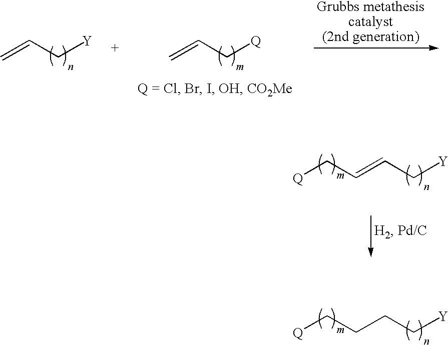

Chain extension by free-radical thiol addition reaction:

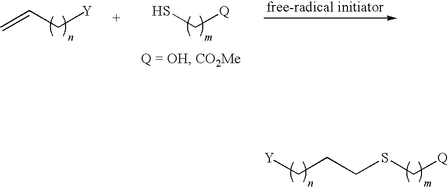

Chain extension by Wittig reaction:

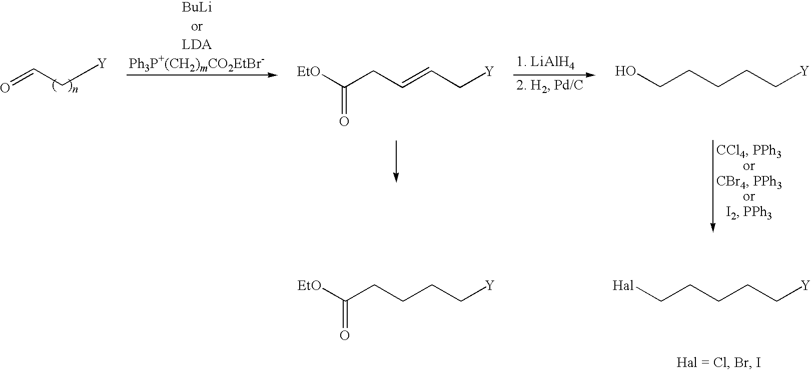

Chain extension by Williamson ether synthesis:

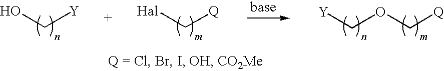

Chain extension by thioether synthesis:

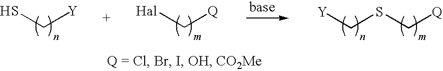

In addition, chain extensions are possible/can be carried out via ester or amide formation.

Further functionalisation to the arylsulfonate (surfactant end product):

Linking of the aromatic compound:

Introduction of sulfonate:

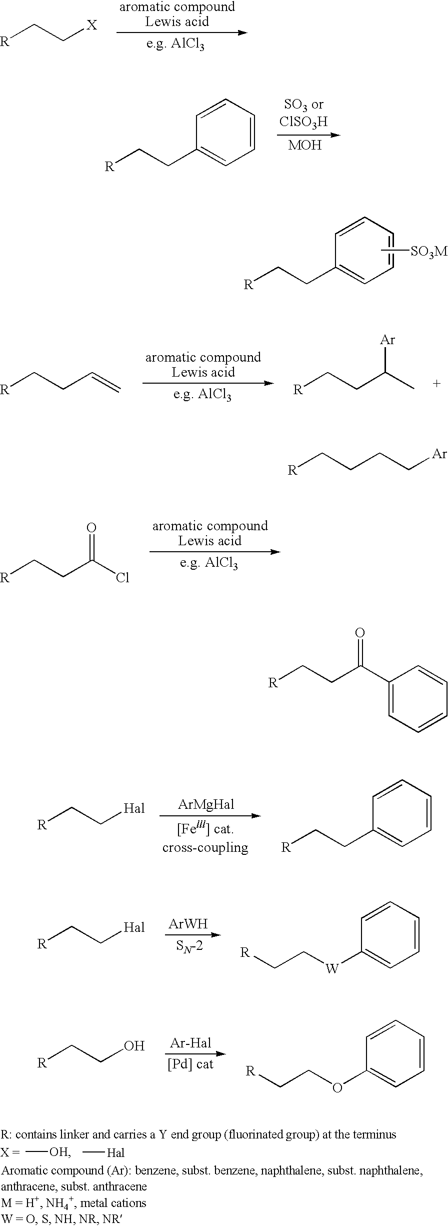

Besides these methods for linking the arylsulfonate, it is also possible to use the linker functionalities shown below (linking of aromatic Y using linkers).

The CF₃—O group can be introduced in accordance with the following schemes:

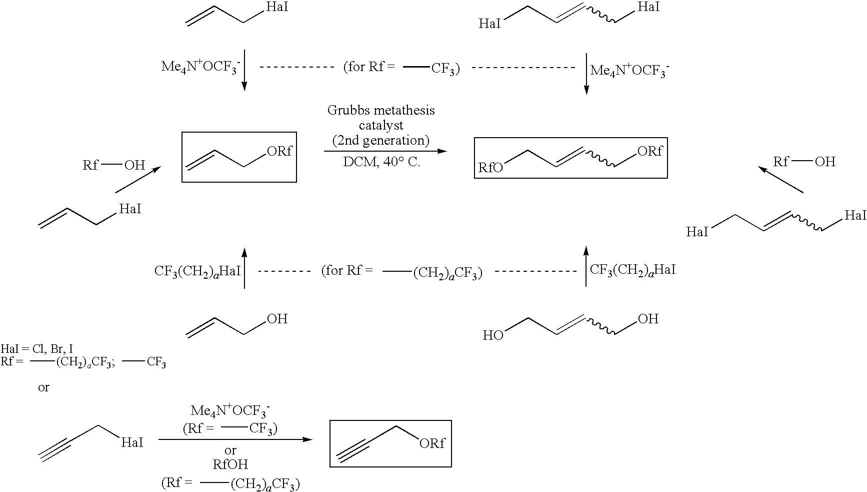

The following applies for Y═CF₃—O:

The aliphatic OCF₃ group can be obtained, for example, from alcohols via the fluorodesulfuration of xanthogenates (K. Kanie, Y. Tanaka, K. Suzuki, M. Kuroboshi, T. Hiyama, Bull. Chem. Soc. Jpn. 2000, 73, 471-484; P. Kirsch, Modern Fluoroorganic Chemistry: Synthesis, Reactivity, Applications, Wiley-VCH, Weinheim, 2004, pp. 67 ff., pp. 144 ff.). The corresponding disclosure of the said method in the cited references thus expressly also belongs to the disclosure content of the present application. The introduction of the arylsulfonate group is possible by methods known to the person skilled in the art.

Variant A:

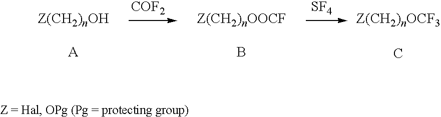

Variant B:

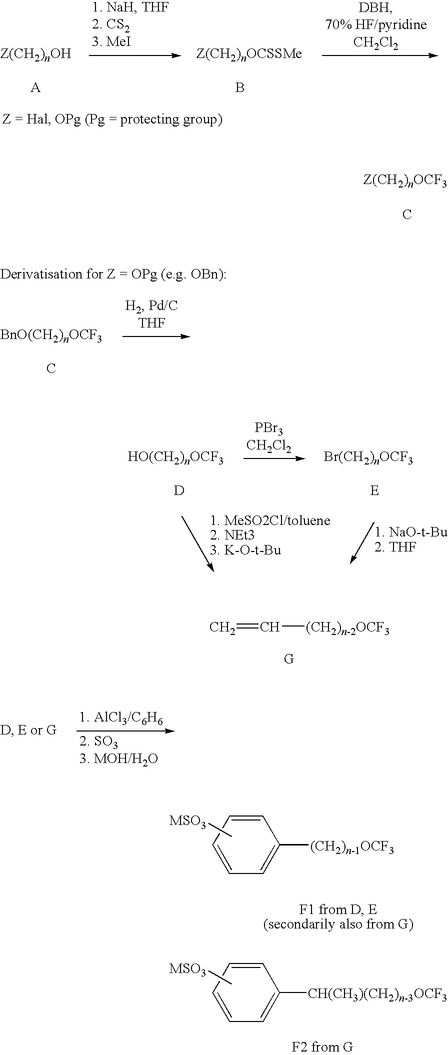

The vinyl compound G can be obtained from compound D or E by elimination.

The following applies for Y═CF₃—(CH₂)ₐ—O—, where a=2:

The CF₃—(CH₂)₂—O— group is introduced by reaction of CF₃—(CH₂)₂—OH with a compound Z—(CH₂)ₙ—Br, where CF₃—(CH₂)₂—OH is commercially available, and Z represents a protected OH group, and subsequent deprotection of the hydroxyl group.

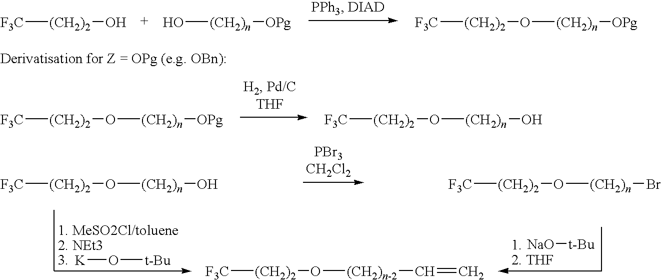

The CF₃(CH₂)ₐS— group can also be introduced analogously via a Mitsunobu reaction or nucleophilic substitution. As shown in the diagrams, analogous transformations give the corresponding compounds (for example with an alcohol, halide or olefin functionality at the terminus).

The SF₅ group can be introduced in accordance with the following schemes:

For alcohols and halides:

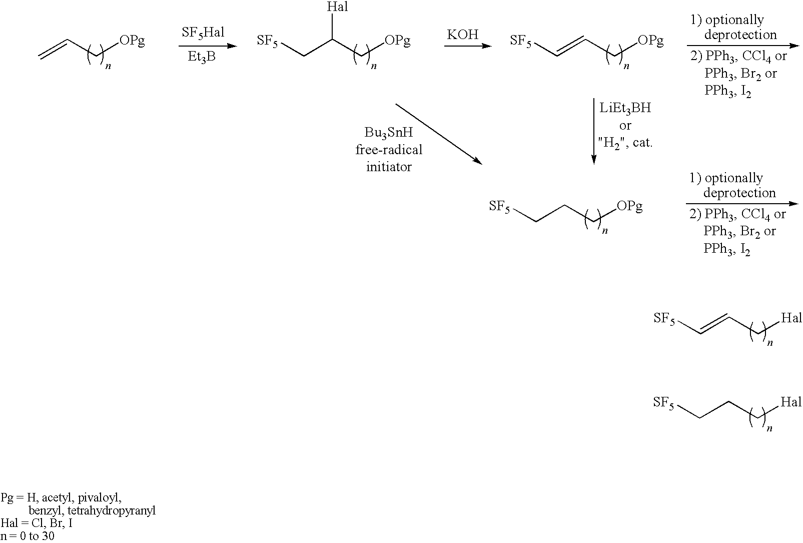

For esters, carboxylic acids and acid chlorides:

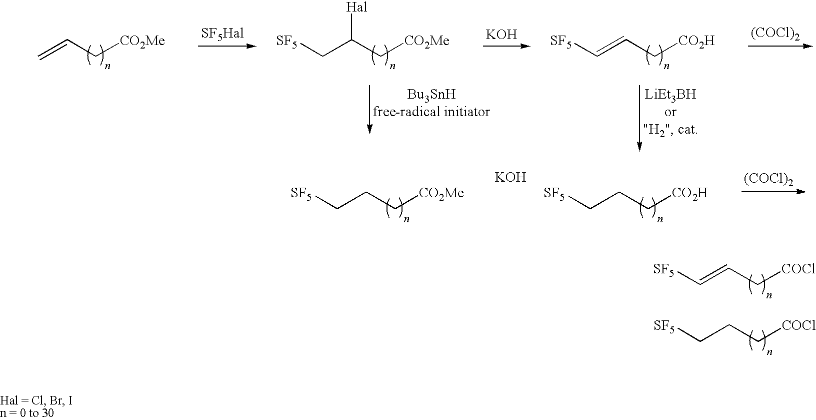

For nitriles, amines, aldehydes and ketones:

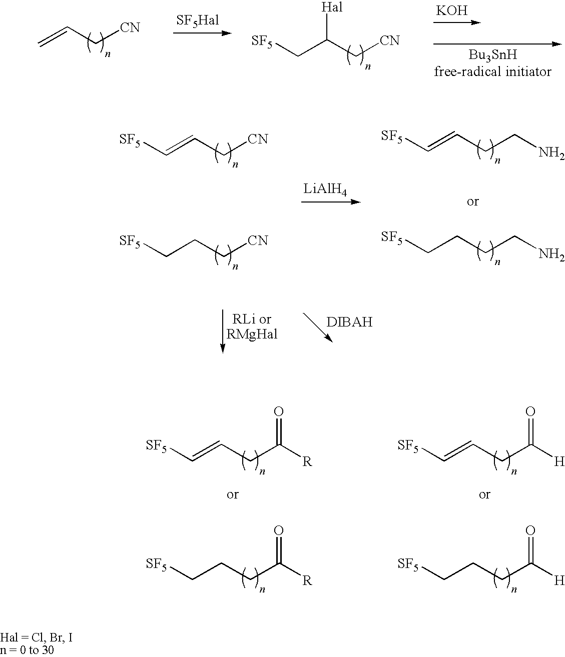

Stepwise or direct oxidation:

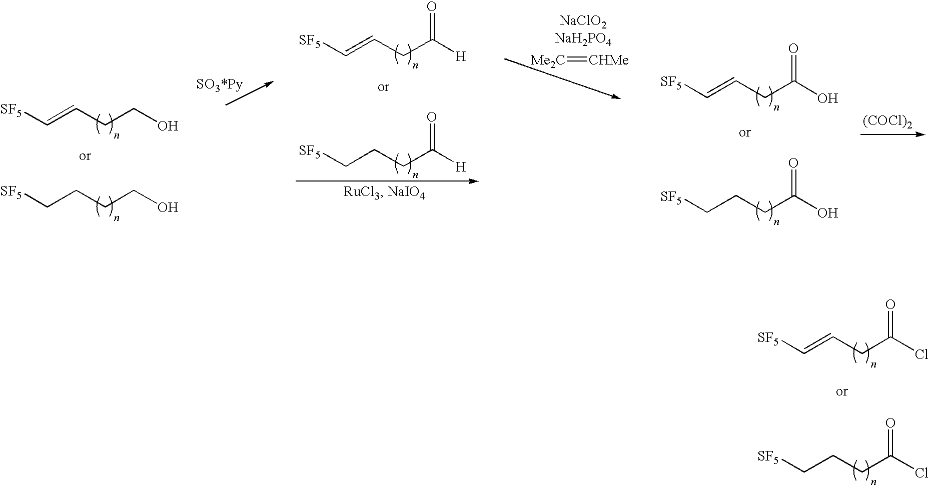

For olefins and alkynes:

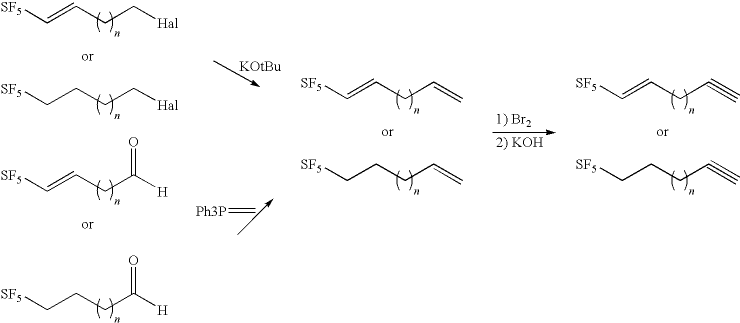

The aliphatic SF₅ group can be introduced, for example, at terminal double bonds via the free-radical addition reaction of SF₅Cl or SF₅Br. A dehydrohalogenation or a hydrogenation, for example, can subsequently optionally be carried out. The first two of these reaction steps are described in the literature (R. Winter, P. G. Nixon, G. L. Gard, D. H. Radford, N. R. Holcomb, D. W. Grainger, J. fluorine Chem. 2001, 107, 23-30), as are catalytic hydrogenations in the presence of an SF₅ function (P. Kirsch, M. Bremer, M. Heckmeier, K. Tarumi, Angew. Chem 1999, 111, 2174-2178; Angew. Chem. Int. Ed. Engl. 1999, 38, 1989-1992). The corresponding disclosure of the said method in the cited references thus expressly also belongs to the disclosure content of the present application. The introduction of the hydrophilic, reactive or polymerisable component is possible via the corresponding ω-SF₅-alcohol by methods known to the person skilled in the art. Examples are revealed by the following scheme:

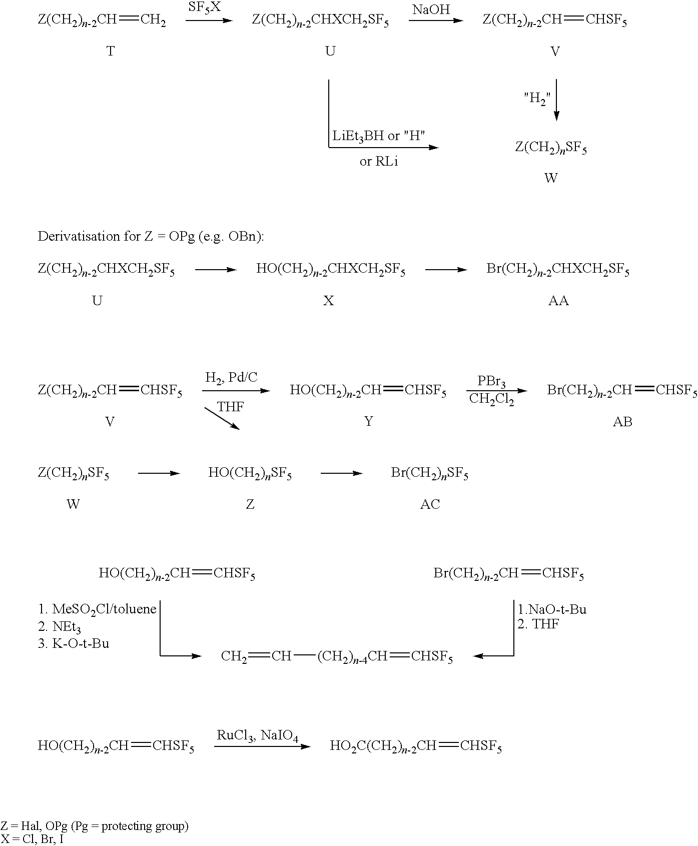

The CF₃—(CH₂)ₐ—S or C₂F₅—S— groups can be introduced in accordance with the following schemes:

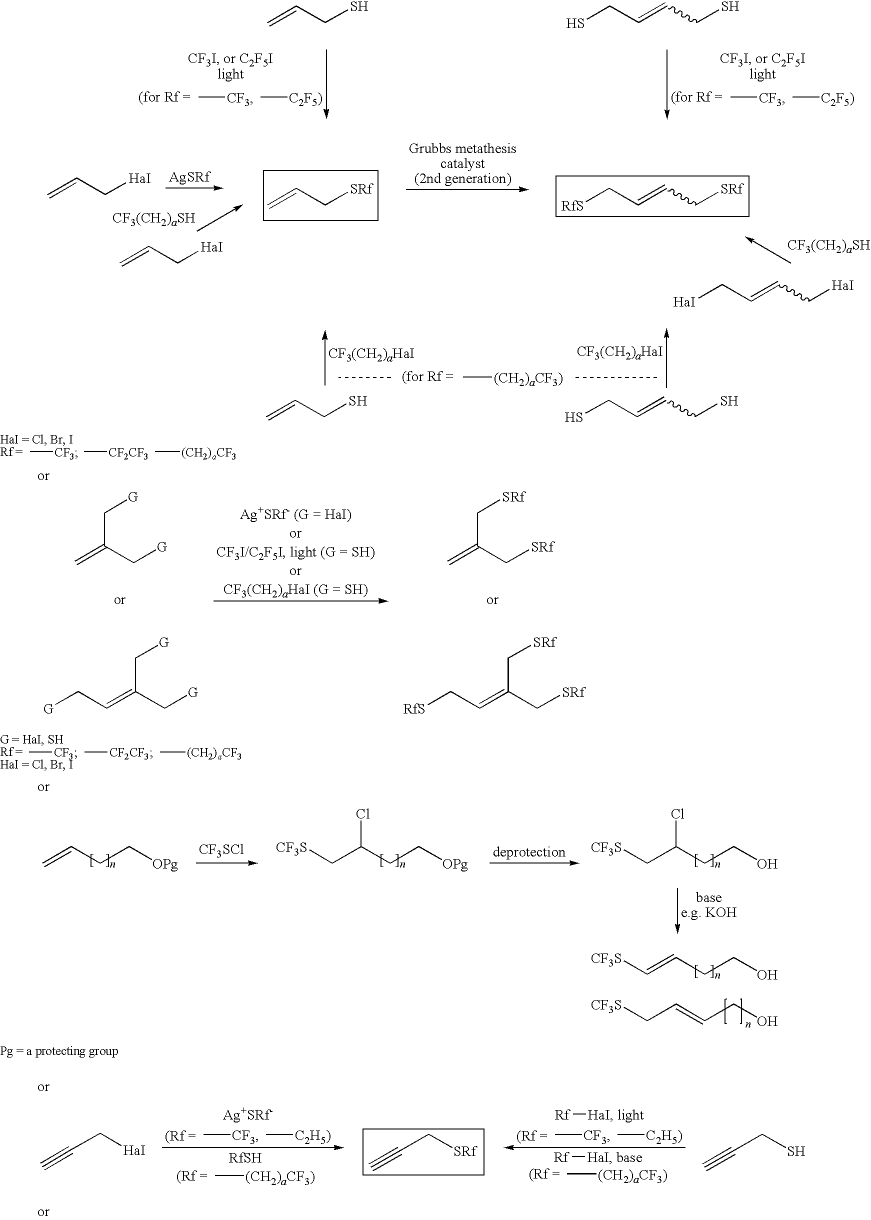

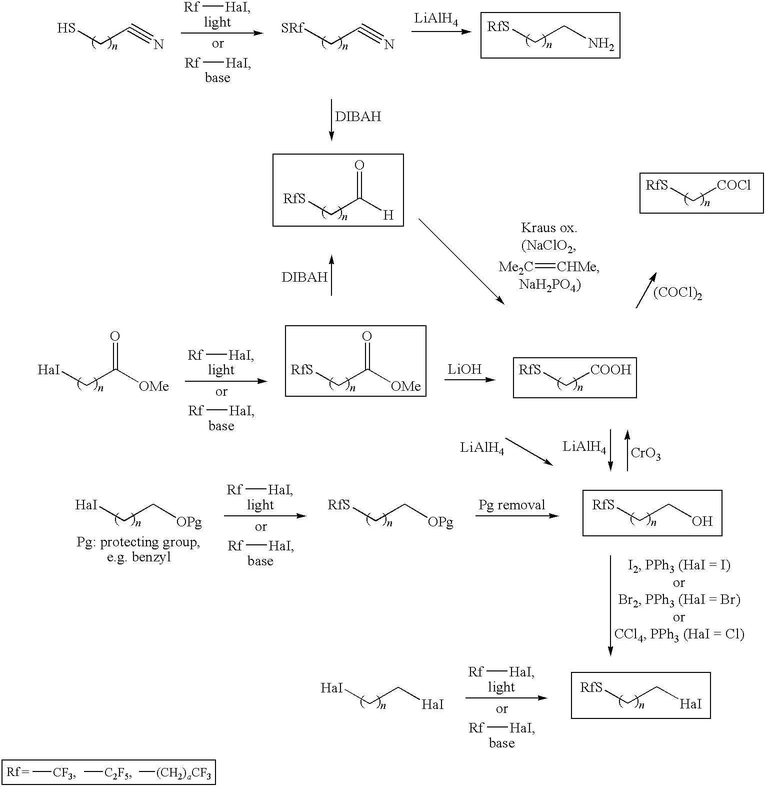

The end groups CF₃(CF₂)_{y}S— where y=0 or 1 can preferably also be introduced into suitable molecules or intermediates by reaction of the corresponding Rf bromide with a thiolate, as described in N. V. Ignatiev et al., Zh. Organich. Khim. 1985, 21(3), p. 653. Direct reaction of the thiol with Rf-Br or Rf-I can also be carried out, as described in N. V. Ignatiev, Ukr. Khim. Zh. 2001, No. 10, pp. 98-102. CF₃S— groups can also be introduced by means of AgSCF₃ as described by N. V. Kondartenko at al. Ukr. Khim. Zh. 1975, 41(6), pp. 516ff. or by means of CF₃SCl as described by W. A. Sheppard, J. Org. Chem. 1964, 29(4), 895ff. The corresponding disclosure of the said method in the cited references thus expressly also belongs to the disclosure content of the present application.

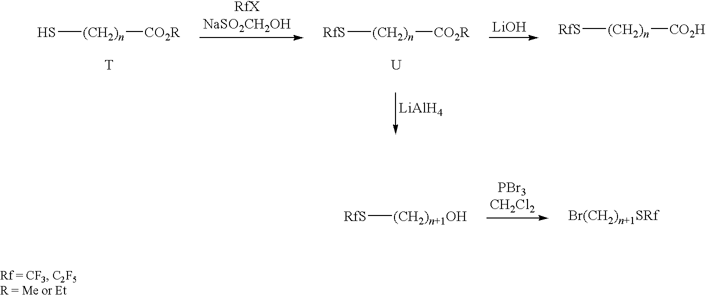

For Y═CF₃—(CH₂)ₐ—S— where a=2:

The CF₃—(CH₂)₂—S— group is introduced by reaction of CF₃—(CH₂)₂—I with a compound Z—(CH₂)ₙ—SH, where Z—(CH₂)ₙ—SH is readily accessible from the corresponding halide, and Z represents a protected OH group, and subsequent deprotection of the hydroxyl group.

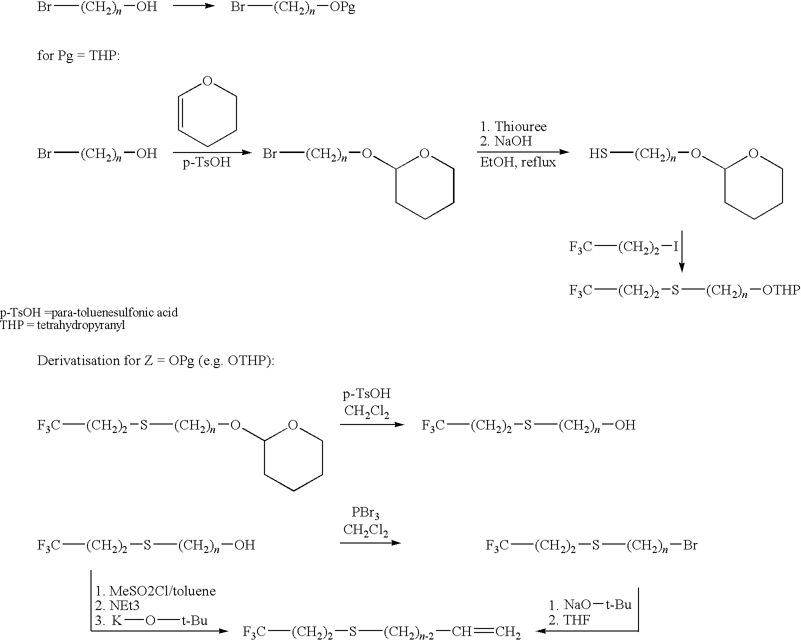

The (CF₃)₂N group can be introduced in accordance with the following schemes:

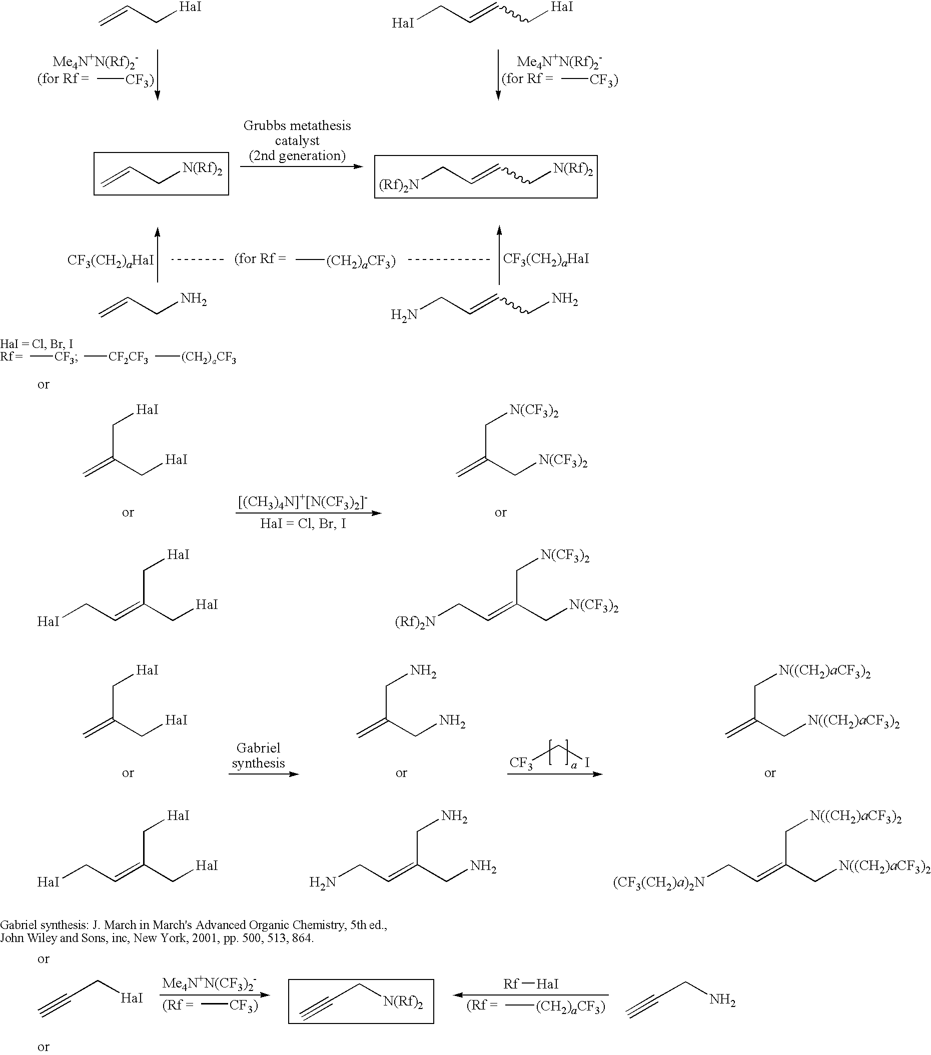

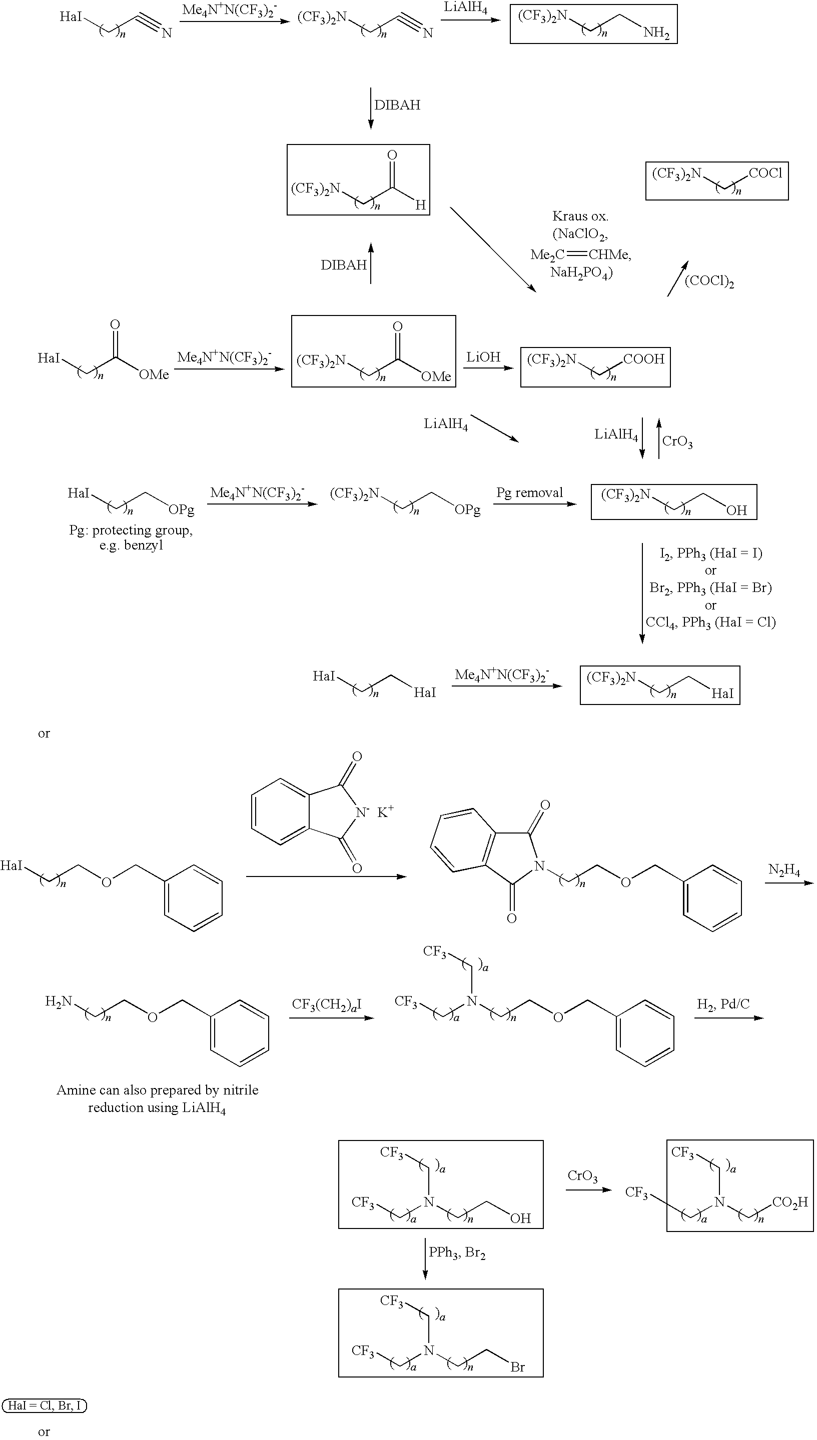

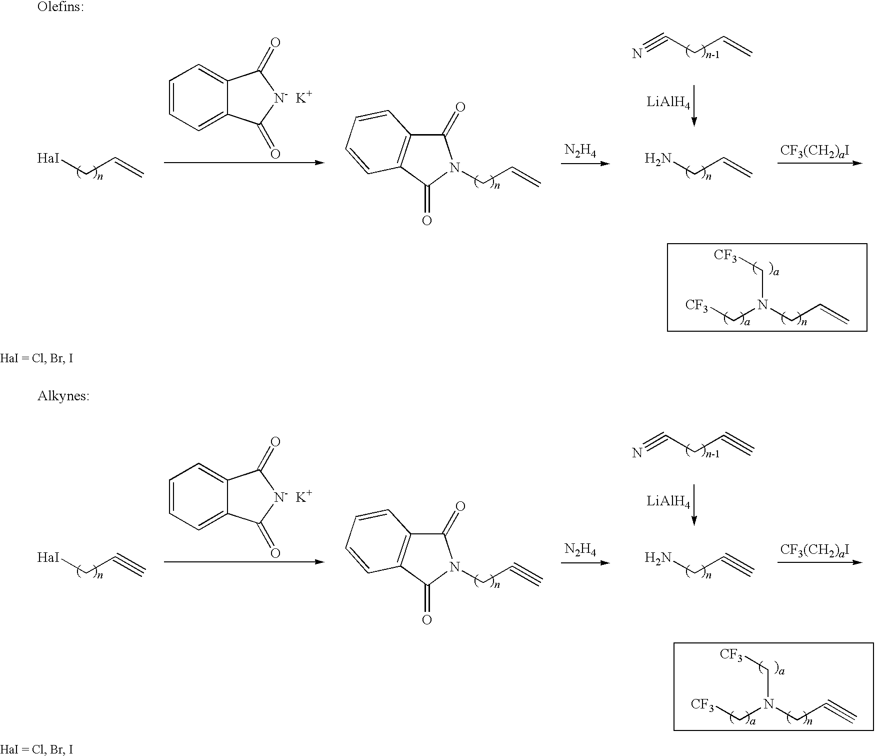

The following applies for Y═(CF₃)₂N—:

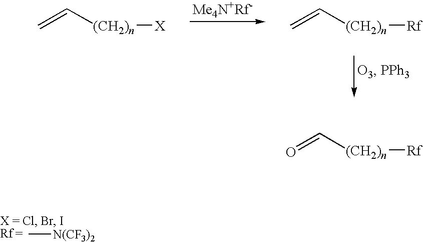

The terminal double bond can be employed immediately in the Friedel-Crafts alkylation. Modification to give the corresponding alcohols or bromides is shown in the following scheme:

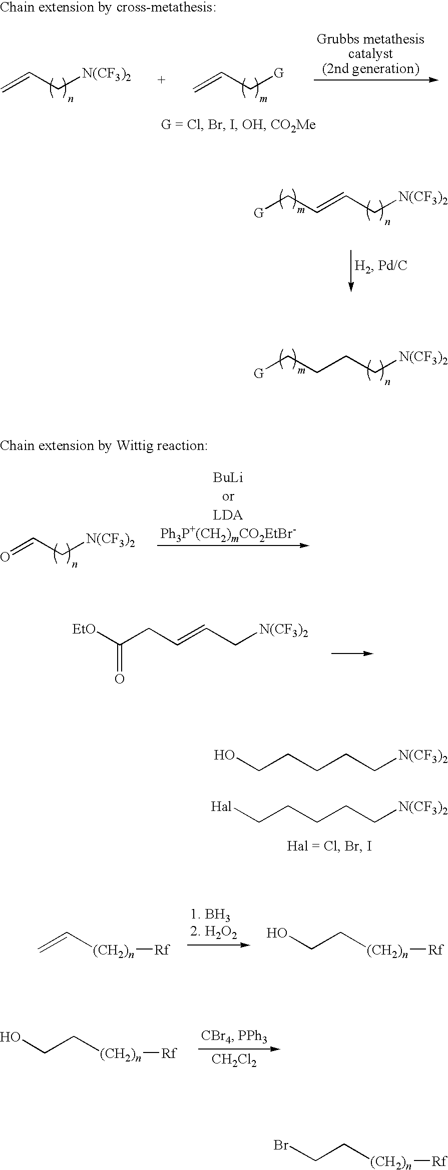

The amine building block [CF₃—(CH₂)ₐ]₂N—, where a stands for an integer selected from the range from 1 to 5, can be introduced with the aid of the Gabriel synthesis (Organikum: Organisch-Chemisches Grundpraktikum [Basic Practical Organic Chemistry], 16th Edn., VEB Deutscher Verlag der Wissenschaften, Berlin, 1986), followed by liberation of the primary amine by reaction with hydrazine. Subsequent alkylation of this amine using CF₃(CH₂)Hal and debenzylation gives the tertiary amino alcohol as key building block.

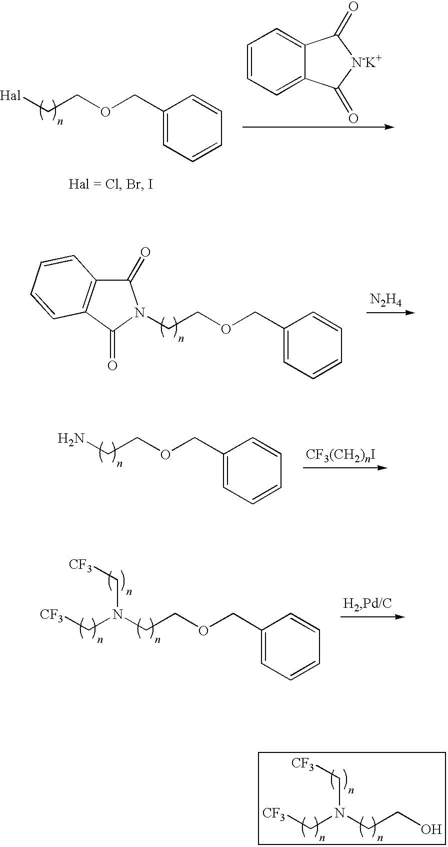

The following applies for Y═CF₃NH—:

The end group CF₃NH— in compounds CF₃NH—R can be introduced by methods known from the literature by reaction of corresponding compounds Cl₂C═N—R with an excess of HF (corresponding syntheses are described, for example, in Petrow et al., Zh. Obshch. Khim. 29 (1959) 2169-2172). Alternatively, it is also possible to react trifluoromethyl isocyanate with an alcohol to give a compound CF₃—NHC(═O)—O—R (as described by Knunyants et al. Mendeleev Chem. J. 22 (1977) 15-105 or Motornyi et al., Zh. Obshch. Khim. 29 (1959) 2157-2122). The corresponding starting materials are each obtainable by methods known from the literature, and the radicals R of the products can be chemically modified by established methods.

The group Y where

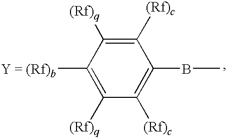

where

Rf=CF₃—(CH₂)ᵣ—, CF₃—(CH₂)ᵣ—O—, CF₃—(CH₂)ᵣS—, CF₃CF₂—S—, SF₅—(CH₂)ᵣ—, [CF₃—(CH₂)ᵣ]₂N—, [CF₃—(CH₂)ᵣ]NH— or (CF₃)₂N—(CH₂)ᵣ—,

B stands for a single bond, O, NH, NR, CH₂, C(O)—O, C(O), S, CH₂—O, O—C(O), N—C(O), C(O)—N, O—C(O)—N, N—C(O)—N, O—SO₂ or SO₂—O

R stands for alkyl having 1 to 4 C atoms,

b stands for 0 or 1 and c stands for 0 or 1,

q stands for 0 or 1, where at least one radical from b and q stands for 1, and

r stands for 0, 1, 2, 3, 4 or 5,

can be introduced in accordance with the following schemes:

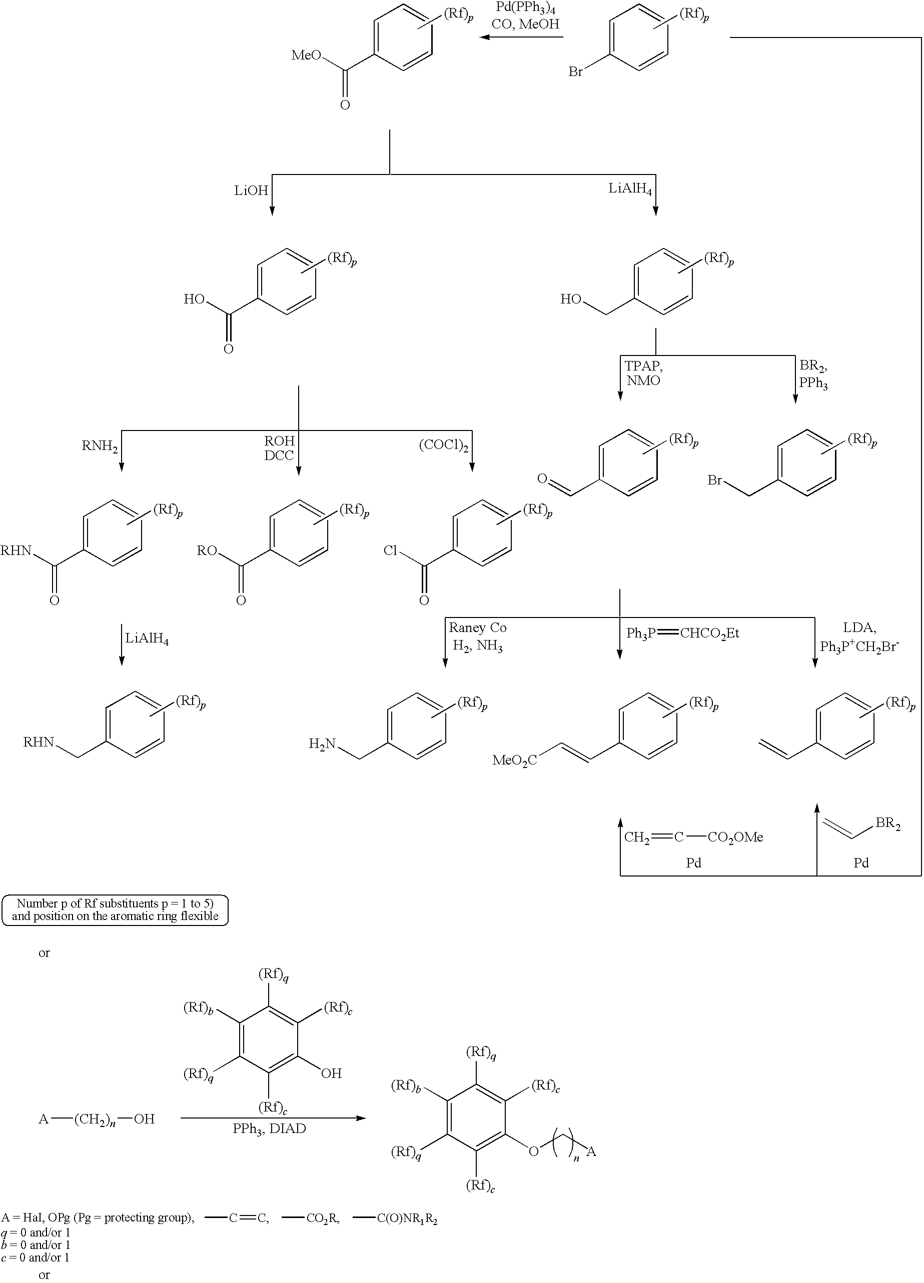

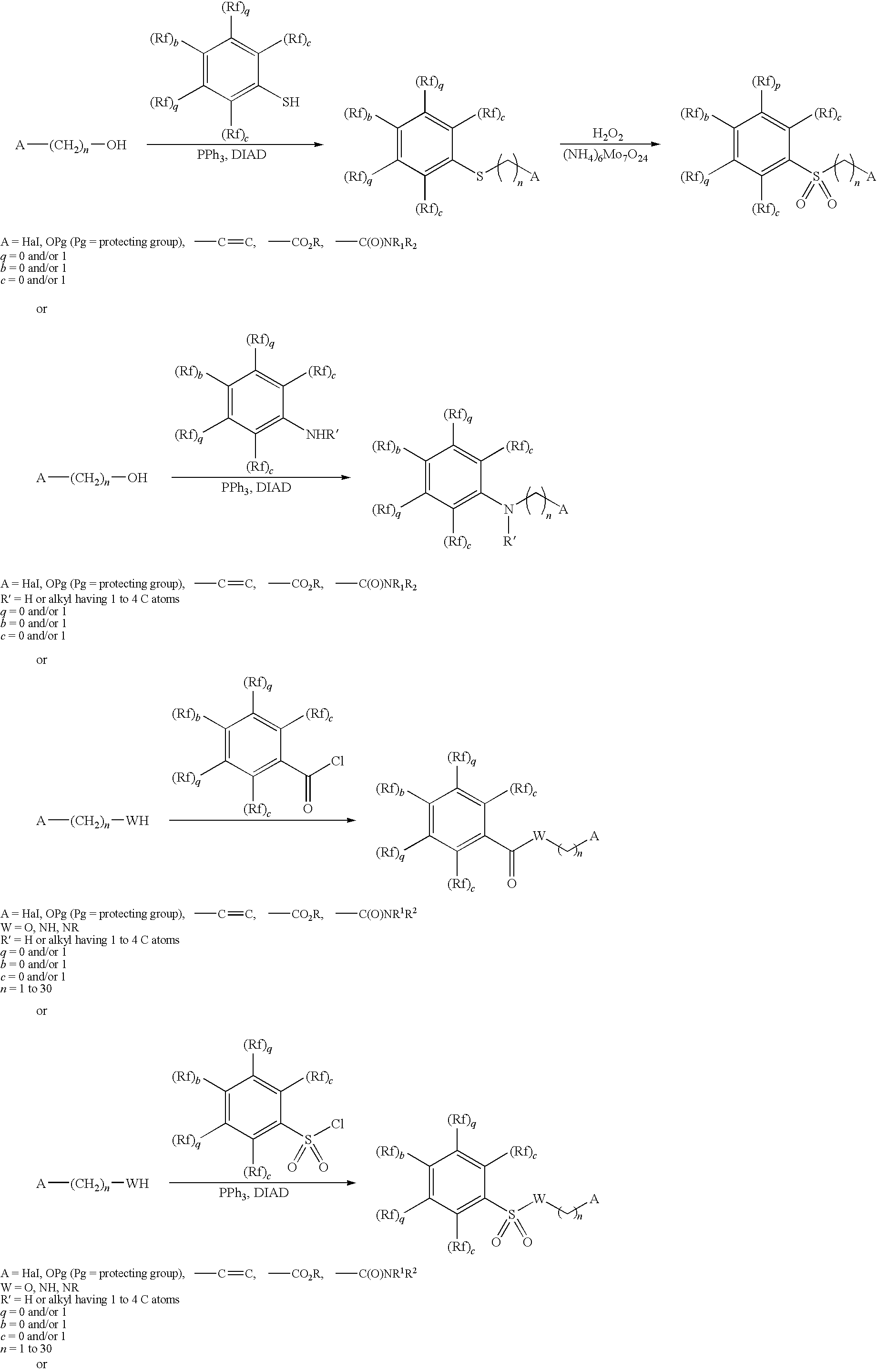

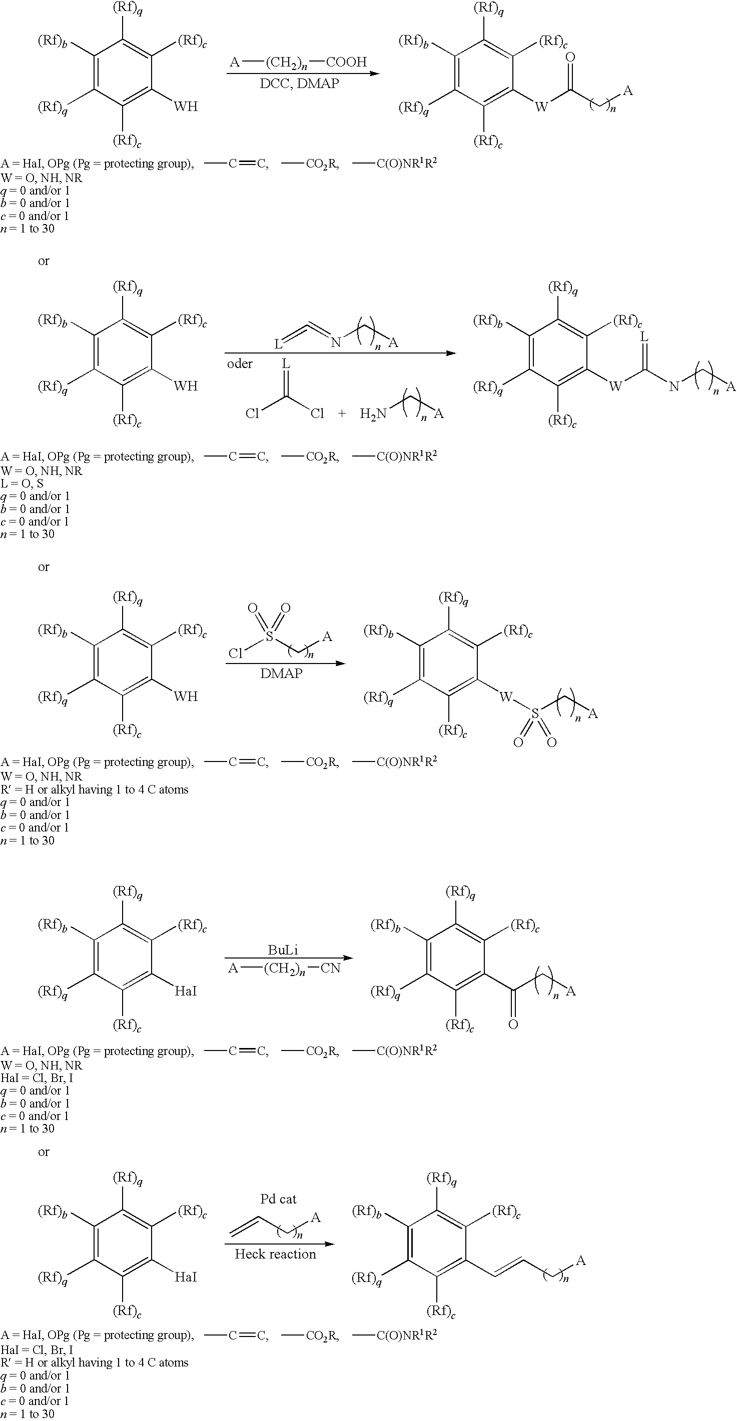

This aromatic group is introduced onto, for example, a diol or alkene in accordance with the scheme shown. In some cases, the respective Rf-substituted aromatic compounds are commercially available. Otherwise, synthetic methods are also indicated in each case. The corresponding disclosure of the said method in the cited references thus expressly also belongs to the disclosure content of the present application.

The group Rf stands for CF₃—(CH₂)ᵣ—, CF₃—(CH₂)ᵣ—O—, CF₃—(CH₂)ᵣ—S—, CF₃CF₂—S—, SF₅—(CH₂)ᵣ—, [CF₃—(CH₂)ᵣ]₂N—, [CF₃—(CH₂)ᵣ]NH— or (CF₃)₂N—(CH₂)ᵣ—, with indices as described above, and can be introduced by means of substitution reactions on aromatic compounds. If Rf is used in the following schemes, the definition given here applies, unless indicated otherwise.

The Rf-substituted aryl building blocks can be introduced by reaction of the corresponding phenols in a Mitsunobu reaction with formation of an ether bridge (O. Mitsunobu Synthesis 1981, 1).

The bonding of the aryl group via an NH or NR bond is likewise carried out under Mitsunobu conditions in accordance with the following scheme:

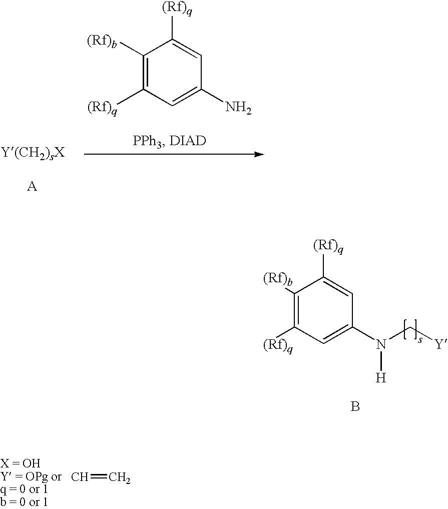

The aryl building blocks containing the said Rf substituents can be synthesised in accordance with the following reactions:

The following applies for CF₃ substitution: the CF₃ groups can be obtained by reaction of aromatic carboxylic acids with HF and SF₄ under super-atmospheric pressure and elevated temperature, as indicated in the following scheme:

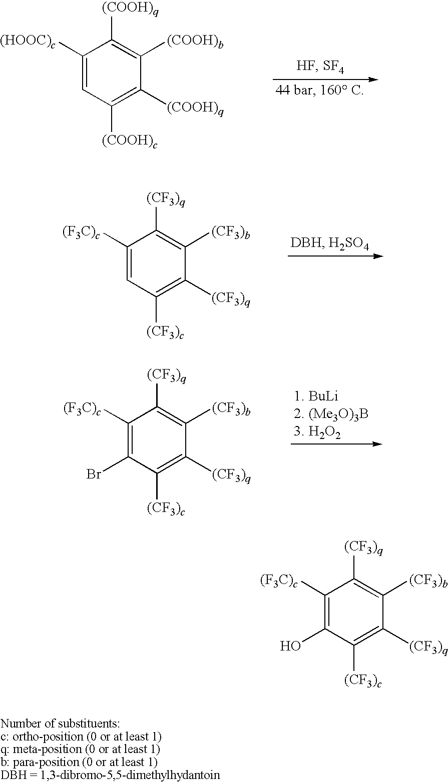

Compounds of the formula

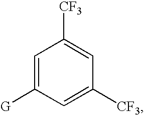

where G=—CO₂H, CH₂NH₂, —CH₂OH, —CHO, —COCl, —CH₂Br, —CH₂CO₂H, —CH═CH₂, —CH═CHCO₂H, —C≡CCH₂OH,

are commercially available.

Derivatisation for aromatic systems containing 3 fluorinated CF₃ groups:

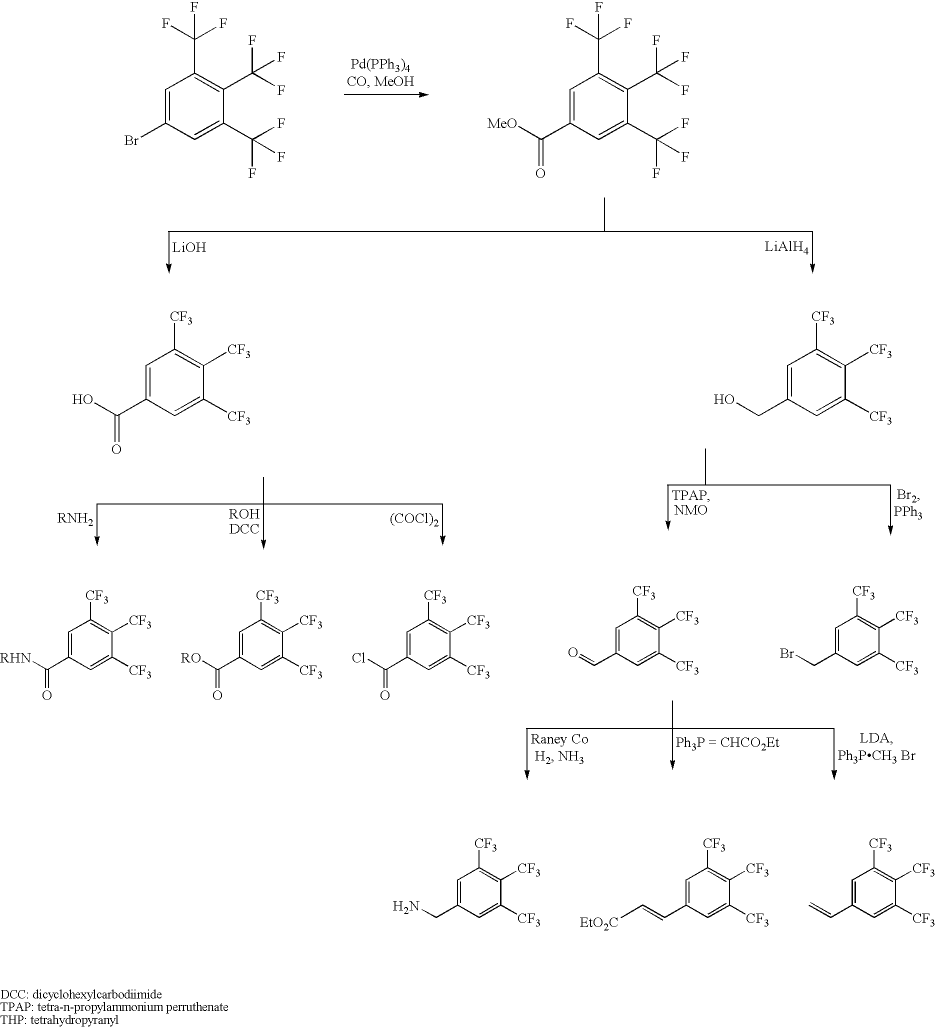

The following applies for SF₅:

The modification of commercial p-nitropentafluorosulfuranyl compounds can be carried out as described in P. Kirsch et al. Angewandte Chemie 1999, 111, 2174-2178.

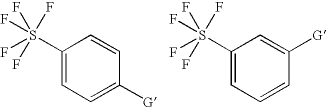

Commercial reagents are: G′=—OH, —Br, —NH₂, —NO₂, —CHO, —CO₂H

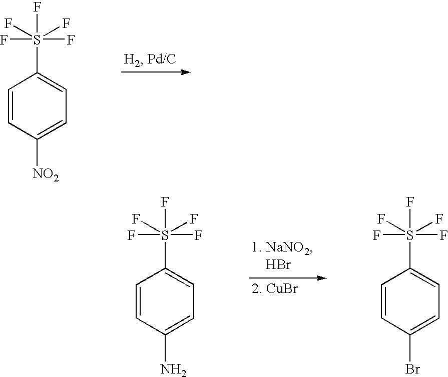

The m,m-bispentafluorosulfuranyl compounds are accessible as described in W. A. Sheppard J. Am. Chem. Soc. 1962, 84, 3064-3072 or U.S. Pat. No. 3,073,861 or U.S. Pat. No. 3,135,736:

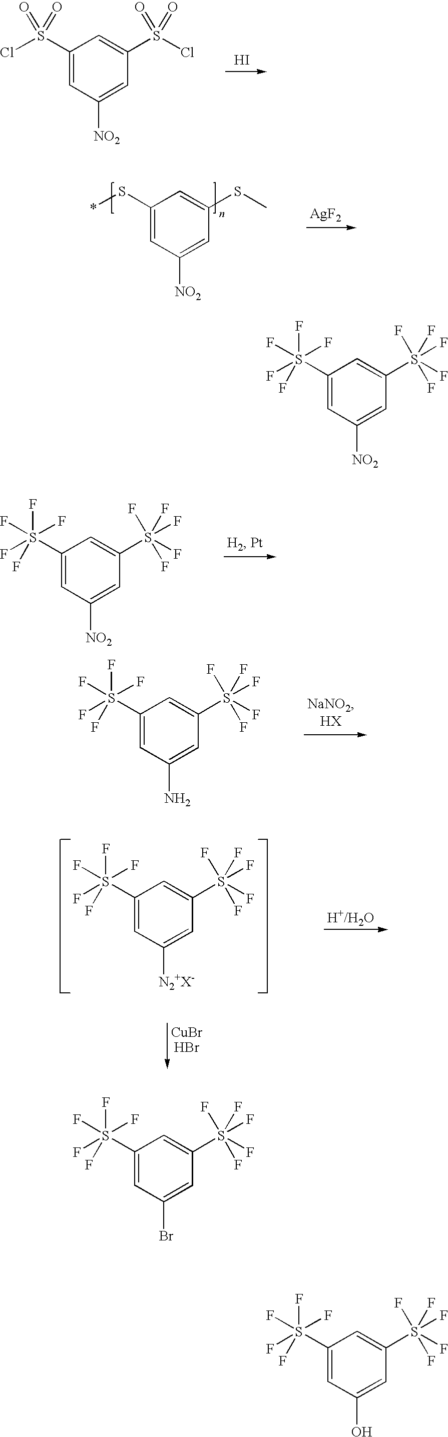

The corresponding disclosure of the said methods in the cited references thus expressly also belongs to the disclosure content of the present application.

The following applies for F₃CS— or F₅C₂S—:

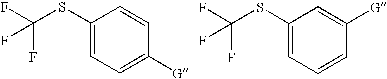

Commercial reagents are:

G″=—OH, —Br, —Cl, —NH₂, —NO₂, —N═C═O, —CHO, —CO₂H, —CN, —CH₂OH, —CH₂Br

Aromatic trifluoromethyl thioethers and pentafluoroethyl thioethers are accessible by substitution of iodoaromatic compounds or etherification of thiophenols, as indicated in the following scheme:

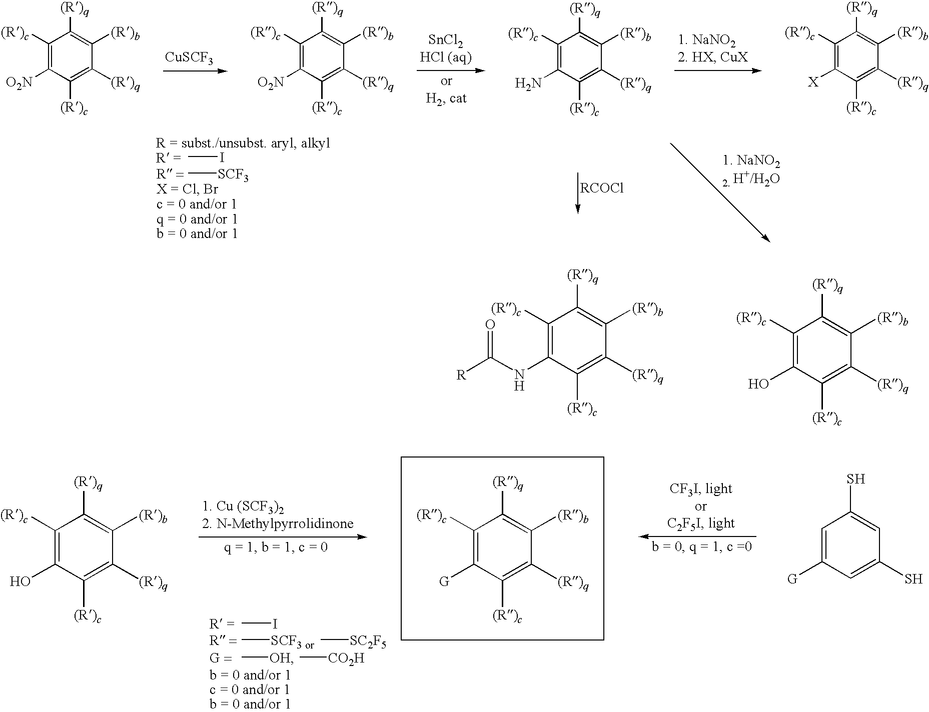

The following applies for F₃CO:

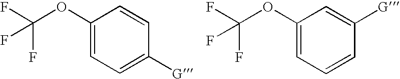

Commercial reagents or substances which are readily accessible therefrom are:

G″=—OH, —I, —Br, —Cl, —NH₂, —SH, —B(OH)₂, —CHO, —CO₂H, —CO₂Me, —CONH₂, —CN, —CH₂OH, —CH₂Br, —CH₂CN.

Trifluoromethoxyaromatic compounds can be obtained by reaction of phenols with carbon tetrachloride and hydrogen fluoride.

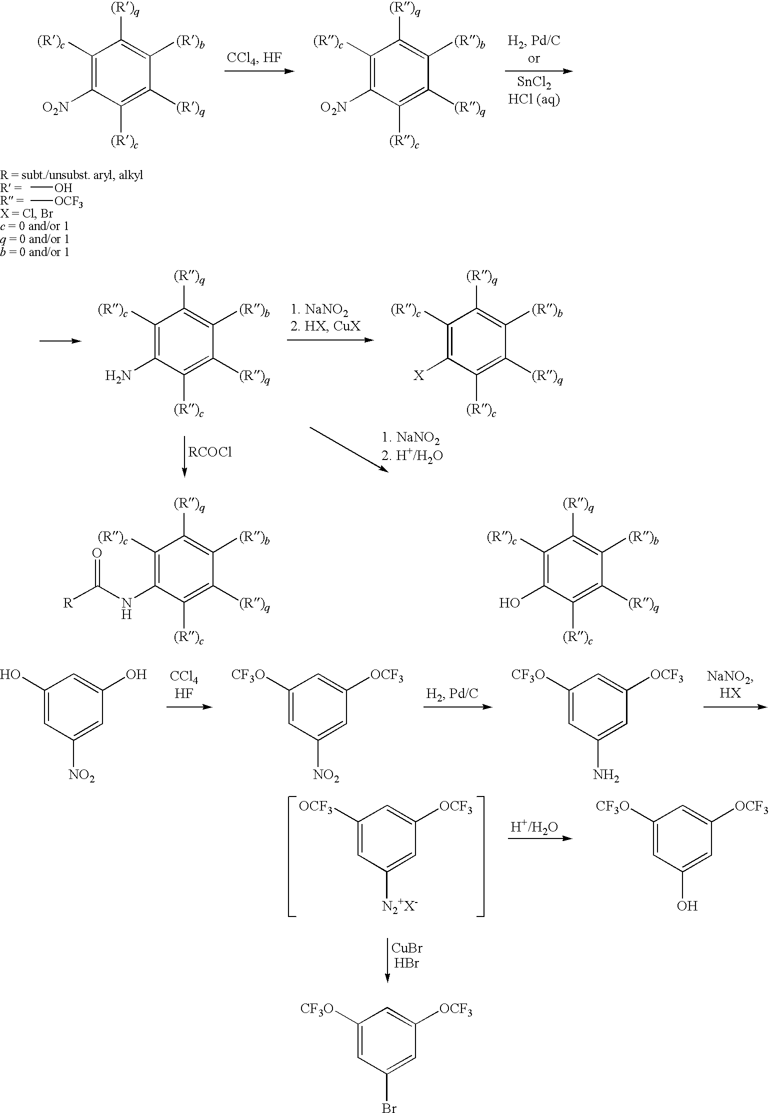

The starting material nitroresorcinol can be prepared in accordance with the following literature:

Ref. 1 Funke; Krucker; BSCFAS; Bull. Soc. Chim. Fr.; 1953; 744, 746.

Ref. 1 Grosheintz; Fischer; JACSAT; J. Am. Chem. Soc.; 70; 1948; 1476, 1478.

The following applies for [CF₃—(CH₂)ₐ]₂N—:

The amine building block [CF₃—(CH₂)ₐ]₂N—, where a stands for an integer selected from the range from 0 to 5, can be introduced with the aid of the Gabriel synthesis (Organikum: Organisch-Chemisches Grundpraktikum [Basic Practical Organic Chemistry], 16th Edn., VEB Deutscher Verlag der Wissenschaften, Berlin, 1986), followed by liberation of the primary amine by reaction with hydrazine. Subsequent alkylation of this amine using CF₃(CH₂)ₐHal and debenzylation gives the tertiary amino alcohol as key building block.

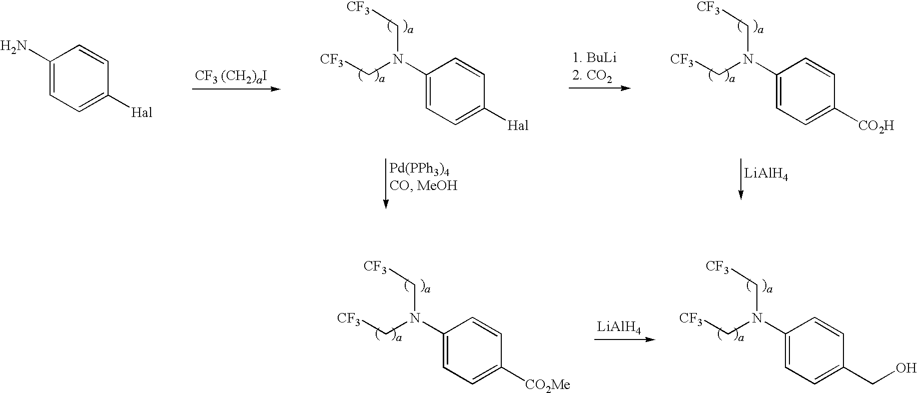

The following applies for (CF₃)₂N—:

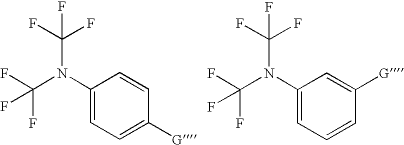

Commercial reagents or substances which are readily accessible therefrom are:

G″″—OH, —I, —Br, —Cl, —NH₂, —NHAc, —CHO, —CO₂H, —CO₂Me, —CONH₂, —CN, —CH₂OH, —CH₂Br, —CH₂CN.

(CF₃)₂N substituents can be obtained as described by F. S. Fawcett; J. Am. Chem. Soc. 84 (No. 22) (1962) 4275-4285 starting from isocyanates by reaction with fluorophosgene and subsequent fluorination using SF₄/HF or starting from isothiocyanates by reaction with mercury difluoride and subsequent reaction with fluorophosgene, and subsequent fluorination using SF₄/HF:

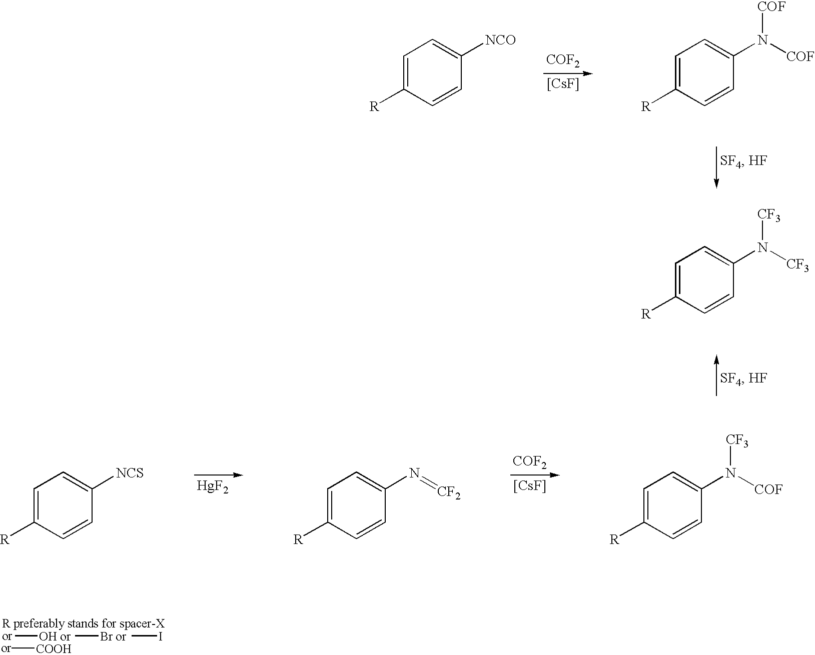

An alternative route for the preparation of the bistrifluoromethylanilines starts from aromatic aldehydes and is described in detail in R. E. Banks, J. Chem. Soc. Perkin Trans. 1 (1973) 80-82:

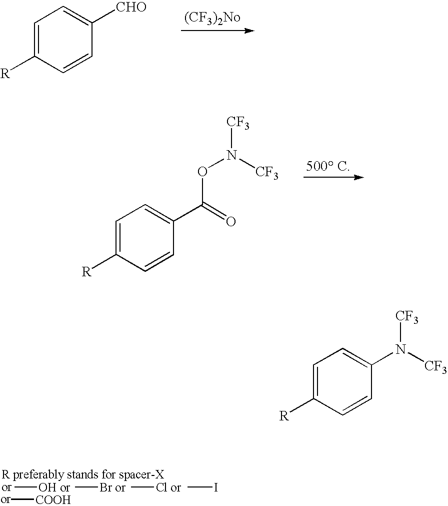

The following applies for CF₃NH—:

The end group CF₃NH— in compounds CF₃NH—R can be introduced by methods known from the literature by reaction of corresponding compounds Cl₂C═N—R with an excess of HF (corresponding syntheses are described, for example, in Petrow et al., Zh. Obshch. Khim. 29 (1959) 2169-2173 or E. Kuhle, Angew. Chem. 89 (No. 11) (1977), 797-804). Alternatively, trifluoromethyl isocyanate can also be reacted with an alcohol to give a compound CF₃—NHC(═O)—O—R (as described by Knunyants et al. Mendeleev Chem. J. 22 (1977) 15-105 or Motornyi et al., Zh. Obshch. Khim. 29 (1959) 2157-2122). The corresponding starting materials are each obtainable by methods known from the literature, or compounds of the Cl₂C═N—R type can be obtained by reactions of compounds R—NH—CHO with chlorine and SOCl₂, and the radicals R of the products can be chemically modified by established methods.

The corresponding disclosure of the said methods in the references cited here thus expressly also belongs to the disclosure content of the present application.

The choice of suitable solvents and reaction conditions presents the person skilled in the art in the case of the said reactions with absolutely no difficulties (Organikum: Organisch-Chemisches Grundpraktikum [Basic Practical Organic Chemistry], 16th Edn., VEB Deutscher Verlag der Wissenschaften, Berlin, 1986).

Apart from the preferred compounds mentioned in the description, the use thereof, compositions and processes, further preferred combinations of the subject-matters according to the invention are disclosed in the claims.

The disclosures in the cited references thus expressly also belong to the disclosure content of the present application.

The following examples explain the present invention in greater detail without restricting the scope of protection. In particular, the features, properties and advantages, described in the examples, of the compounds on which the particular examples are based can also be applied to other substances and compounds which are not mentioned in detail, but fall within the scope of protection, so long as nothing to the contrary is stated elsewhere. In addition, the invention can be carried out throughout the claimed range and is not restricted to the examples mentioned here.

### EXAMPLES

List of abbreviations used:

Bn: benzyl

DBH: 1,3-dibromo-5,5-dimethylhydantoin

DCM: dichloromethane

DMAP: 4-(dimethylamino)pyridine

Me: methyl

MTBE: methyl tert-butyl ether

RT room temperature (20° C.)

THF: tetrahydrofuran

PE: petroleum ether

DCC N,N′-dicyclohexylcarbodiimide

TPAP tetra-n-propylammonium perruthenate

9-BBN 9-borabicyclo[3.3.1]nonane

### Example 1

1. Synthesis of the Alcohol

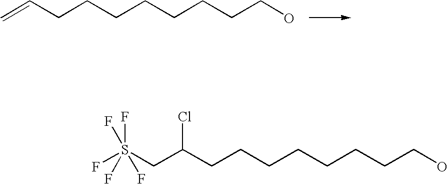

15 g of decenol are dissolved in 250 ml of DCM and cooled to −40° C. 27 g of SF₅Cl— previously condensed by cold trap—are passed into the apparatus as a gas. For activation, 2 ml of 1 M Et₃B solution are added. The activation is repeated until the batch no longer warms during the introduction of gas. The mixture is stirred at the same temperature for a further two hours. The reaction mixture is hydrolysed by addition to ice/NaHCO₃ solution (saturated), then adjusted to pH 10 using NaOH. The aqueous phase separated off is washed twice with MTB ether. The collected organic phases are extracted once with NaCl solution, dried over sodium sulfate and filtered, and the solvent is removed by distillation.

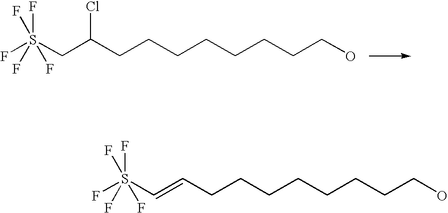

9 g of the starting material are dissolved in 120 ml of ethanol in a 250 ml one-necked flask with reflux condenser, and KOH powder is subsequently added. The reaction mixture is stirred overnight. The reaction mixture is evaporated, and water and MTBE are added, the phases are separated, the aqueous phase is extracted 3 times with MTBE, the collected org. phases are washed with sat. NaCl, dried over Na₂SO₄ and filtered, and the solvent is removed by distillation.

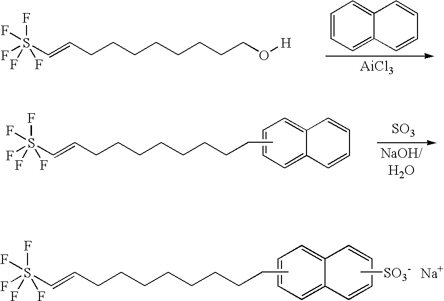

2. Friedel-Crafts Alkylation:

The alcohol (28 g) is dissolved in 200 g of chlorobenzene, 13 g of naphthalene are added, and 13 g of AlCl₃ are subsequently added. After warming at 100° C. for 12 hrs, the reaction mixture is added to ice, and the organic phase is separated off. The org. phase is evaporated, and the alkylnaphthalene is recrystallised.

3. Conversion into the Sulfonate

The alkylnaphthalene (39 g) is sulfonylated using sulfur trioxide (8 g) at a temperature below 40° C. without solvent and isolated. The isolated product is added to a mixture of NaOH (6 g) and water (30 g), giving the sulfonate.

### Example 2

Synthesis of Bromide:

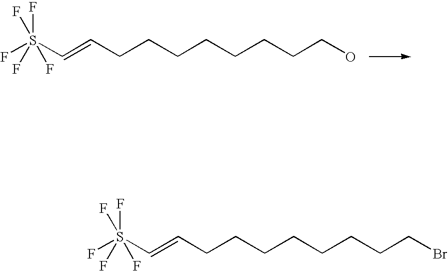

1. Bromination

The alcohol (8.3 g, 29.4 mmol) is initially introduced in dry DCM (0.2 molar solution), and triphenylphosphine (11.6 g, 44 mmol, 1.4 eq) and subsequently, in portions, tetrabromomethane (CBr₄: 16.6 g, 50 mmol, 1.5 eq) are added. The reaction is stirred for 4 hrs and then quenched using sat. NaHCO₃ soln., extracted with MTBE and dried over sodium sulfate. The crude product formed is passed through a column with heptane, giving 9 g of colourless oil.

2. Friedel-Crafts Alkylation:

The bromide (34 g) is dissolved in 200 g of chlorobenzene, 13 g of naphthalene are added, and 13 g of AlCl₃ are subsequently added. After warming at 100° C. for 12 hrs, the reaction mixture is added to ice, and the organic phase is separated off. The org. phase is evaporated, and the alkylnaphthalene is recrystallised.

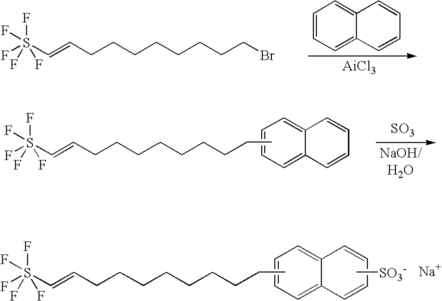

3. Conversion into the Sulfonate

The alkylnaphthalene (39 g) is sulfonylated using sulfur trioxide (8 g) at a temperature below 40° C. without solvent and isolated. The isolated product is added to a mixture of NaOH (6 g) and water (30 g), giving the sulfonate.

4. Analogous Surfactant with Benzene

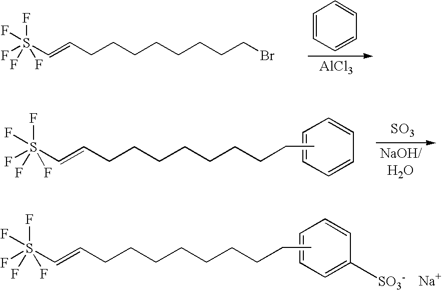

1. (E)-10-bromo-1-pentafluorosulfanyldec-1-ene (35 g) is converted into the alkylbenzene in C₆H₆ (200 g) with AlCl₃ catalysis (26 g).

2. In the second step, (E)-10-(pentafluoro)sulfanyldec-9-enyl)benzene (34 g) is sulfonylated using sulfur trioxide (16 g). The salt formation is carried out using NaOH/water (4 g/20 g).

### Example 3

Step 1:

Synthesis of pentafluorothiobromodec-1-ene

The thiodecanenol—precursor of the thiodecanenyl bromide—is obtained by addition of SF₅Cl onto 10-bromodec-1-ene and subsequent elimination of HCl (see Example 2).

Olefin—Variant 1

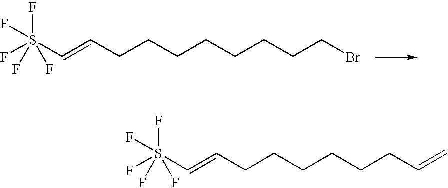

1) K—O-t-Bu (0.12 mol) is added to tetrahydrofuran (75 g), pentafluorothio-bromodec-1-ene (0.1 mol) is then metered in, and the mixture is stirred at 50° C. for 12 hrs. The reaction mixture is added to ice. The (E)-1-penta-fluorosulfanyldeca-1,9-diene building block is isolated and purified using conventional laboratory methods.

Olefin—Variant 2

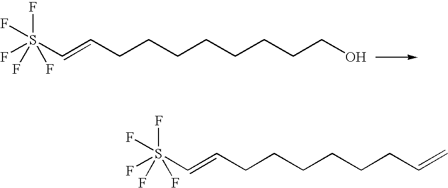

The alcohol (0.1 mol) is converted into the methanesulfonic acid ester intermediate using MeSO₂Cl (0.11 mol) and 0.15 mol of NaHCO₃ in THF (75 g) at 10-20° C. (reaction time: 1-3 hrs). Na—O-t-Bu (0.15 g) is added to the reaction mixture, which is then stirred at 25° C. for 6 hrs. The reaction mixture is added to ice. (E)-1-pentafluorosulfanyldeca-1,9-diene is isolated and purified using conventional laboratory methods.

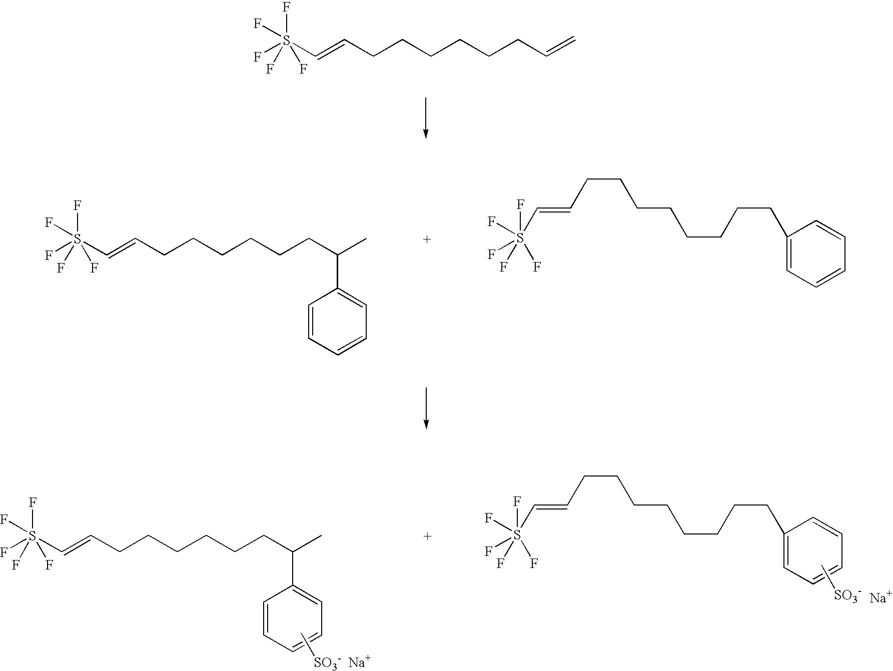

1) (E)-1-pentafluorosulfanyldeca-1,9-diene (26 g) is initially introduced in benzene (120 g) and converted into the corresponding alkylbenzene with AlCl₃ catalysis (16 g). For isolation, the mixture is quenched using ice, and the organic phase is separated off, filtered and evaporated.

2) The alkylbenzene (34 g) is sulfonylated using sulfur trioxide (17 g). For salt formation, the mixture is added to ice/methyl tert-butyl ether, the water phase is extracted with this solvent, and all org. phases are evaporated. Ethanol, then NaOH (4 g) are added, the mixture is warmed briefly and cooled, and the crystals formed are isolated and dried.

### Example 4

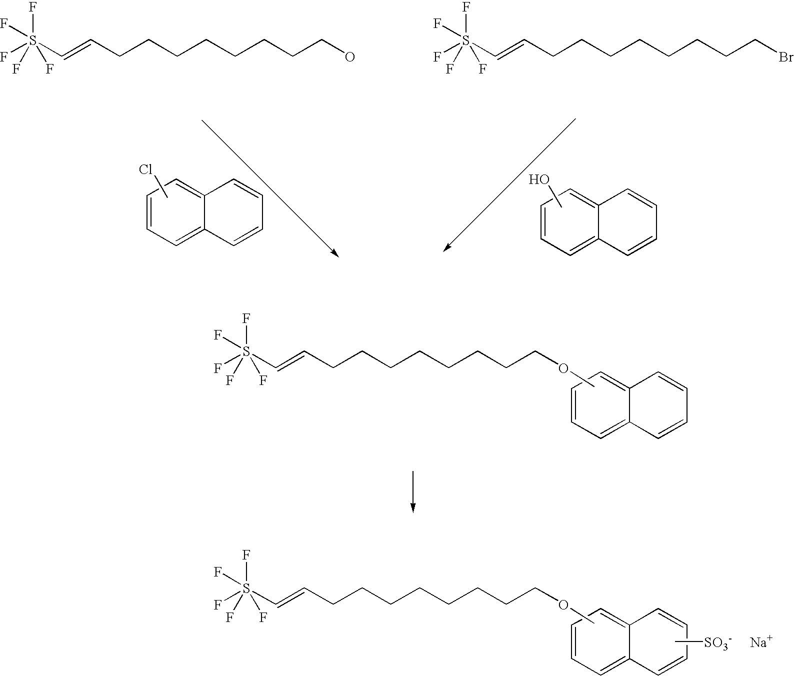

1) Alcohol (28 g), prepared in accordance with Example 1, base Na—O-t-Bu (0.2 g) and catalyst Pd-DBA (5 g) are added to 2-(3-)chloronaphthalene (16 g) in toluene (100 g). After warming (6 hrs; 100° C.) and cooling, the alkoxynaphthalene is isolated using conventional laboratory methods.

2) Bromo compound (35 g), prepared in accordance with Example 2, and base Na₂CO₃ (11 g) are added to 2-(3-)hydroxynaphthalene (15 g) in toluene (100 g). After warming (3 hrs; 80° C.) and cooling, the alkoxynaphthalene is isolated using conventional laboratory methods.

3) The alkoxynaphthalene (41 g) is sulfonylated using sulfur trioxide (12 g) without solvent (temp. below 40° C.)/then, after isolation, salt formation using a mixture of NaOH (6 g) and water (30 g).

### Example 5

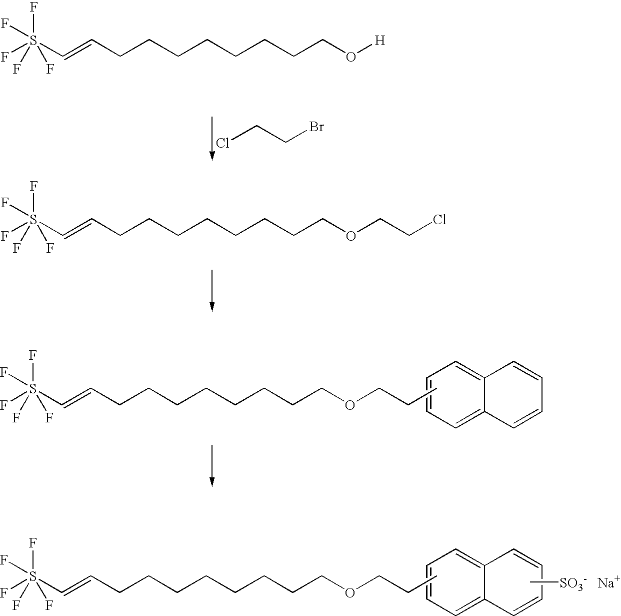

1) The hexenol (28 g), prepared in accordance with Example 1, is etherified using bromochloroethane (14 g) in 200 ml of THF with addition of triethylamine (10 g) at 40° C. The product is isolated using conventional laboratory methods.

2) The chloroethyl ether (35 g) and naphthalene (13 g) are subsequently converted into alkylnaphthalene with AlCl₃ catalysis (26 g). The product is likewise isolated using conventional laboratory methods.

3) The alkylnaphthalene (44 g) is sulfonylated using sulfur trioxide (17 g) and, for salt formation, added to ice/methyl tert-butyl ether, the water phase is extracted with this solvent, and all organic phases are evaporated. Firstly Ethanol, then NaOH (4 g) are added, the mixture is warmed briefly and cooled, and the crystals formed are isolated and dried.

### Example 6

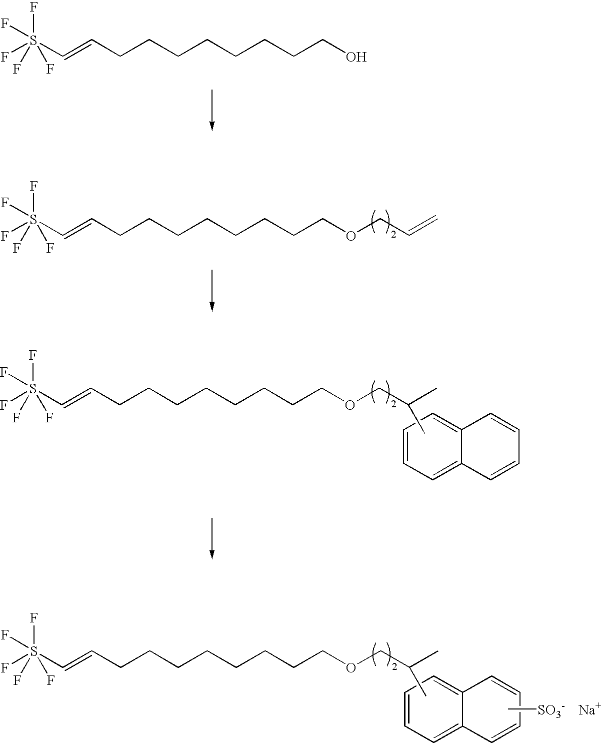

1) The alcohol (28 g), prepared in accordance with Example 1, is stirred at 50° C. for 4 hrs with the homoallyl chloride (0.1 mol) and Na₂CO₃ (11 g) in THF (100 g), and the homoallyl ether is then isolated using conventional laboratory methods.

2) The homoallyl ether (0.1 mol) is dissolved in chlorobenzene (100 g), naphthalene (13 g) and AlCl₃ (26 g) are added, and the mixture is stirred at 80° C. for 6 hrs, and the alkylnaphthalene is then isolated using conventional laboratory methods.

3) The alkylnaphthalene (45 g) is converted into the alkylnaphthalene-sulfonic acid using sulfur trioxide (12 g). For salt formation, the mixture is added to ice/methyl tert-butyl ether, the water phase is extracted with this solvent, and all org. phases are evaporated. Ethanol, then NaOH (4 g) are added, the mixture is warmed briefly and cooled, and the crystals formed are isolated and dried.

### Example 7

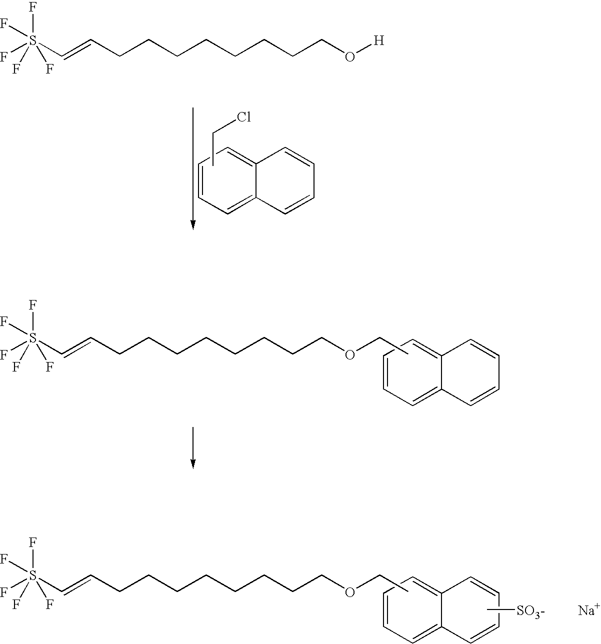

1) Analogously to Example 3, 28 g of alcohol, prepared in accordance with Example 1, are reacted with 18 g of 2-(3)-chloromethylnaphthalene in the presence of 0.2 g of Na—O-t-Bu and 5 g of Pd-DBA.

2) The alkoxymethylnaphthalene (42 g) is sulfonated using SO₃ (12 g) and, for salt formation, added to ice/methyl tert-butyl ether. The water phase is extracted with this solvent, and all org. phases are evaporated. Ethanol, then NaOH (4 g) are added, the mixture is warmed briefly and cooled, and the crystals formed are isolated and dried.

### Example 8

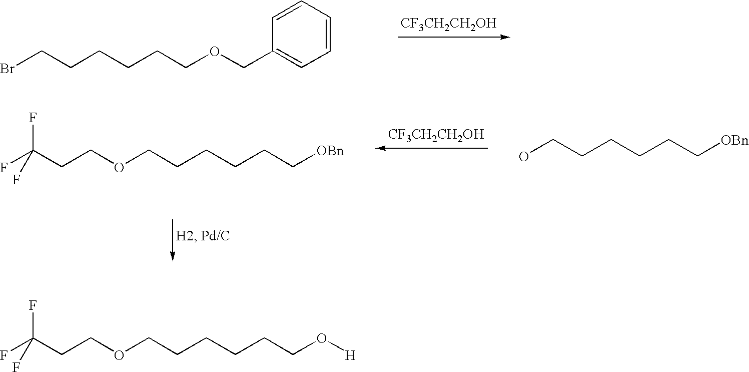

1) Etherification/variant 1. The bromohexane (27 g) and Na₂CO₃ (14 g) are added to 3,3,3-trifluoropropan-1-ol (11 g) in THF (50 g), and the mixture is stirred at 50° C. for 6 hrs. Isolation using conventional laboratory methods gives the ether.

2) Etherification/variant 2. The hexanol (21 g) and triphenylphosphine (60 g) are added to 3,3,3-trifluoropropanol (11 g) in THF (50 g), and, for adequate mixing, the mixture is placed in an ultrasound bath for 10 minutes. Diisopropyl azodicarboxylate (DIAD; 50 g) is added dropwise over the course of 15 minutes, and the reaction mixture is stirred vigorously for a total of 3 hrs. The solvent is evaporated. Cold n-hexane (90 ml) is subsequently added, during which triphenylphosphine oxide precipitates out. The solid is filtered off, the solution is evaporated, the residue is taken up in di-chloromethane (100 g), and the solution is filtered through a layer of SiO₂. The eluate is evaporated, and the residue consists of the product.

3) Debenzylation. [6-(3,3,3-Trifluoropropoxy)hexyloxymethyl]benzene (5 g) is taken up in ethanol (30 g) and hydrogenated at 30° C. with addition of catalyst Pd/C (5%) (0.5 g) and hydrogen (atmospheric pressure). The reaction solution is filtered and evaporated, giving the product 6-(3,3,3-trifluoro-propoxy)hexan-1-ol.

4) Friedel-Crafts Alkylation

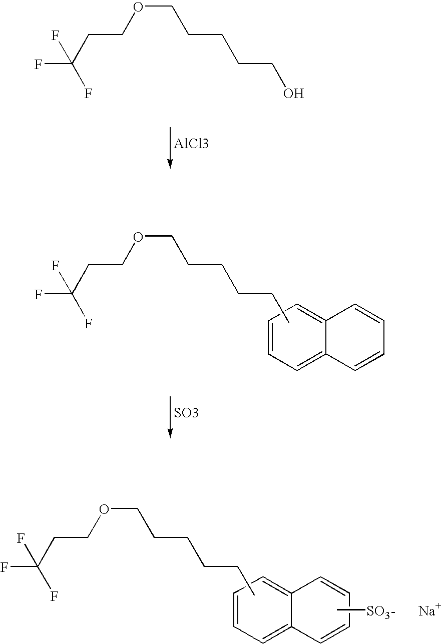

1) Alkylnaphthalene. The alcohol (20 g) is dissolved in chlorobenzene (100 g), and naphthalene (13 g) and then AlCl₃ (26 g) are added. After the mixture has been warmed at 100° C. for 12 hrs, it is added to ice, and the organic phase is separated off. It is filtered and evaporated, and the alkyl-naphthalene is recrystallised.

2) Alkylnaphthalenesulfonic Acid

The alkylnaphthalene (31 g) is sulfonylated using sulfur trioxide (12 g) without solvent (temp. below 40° C.). For salt formation, the mixture is added to ice/methyl tert-butyl ether, the water phase is extracted with this solvent, and all org. phases are evaporated. Ethanol, then NaOH (4 g) are added, the mixture is warmed briefly and cooled, and the crystals formed are isolated and dried.

### Example 9

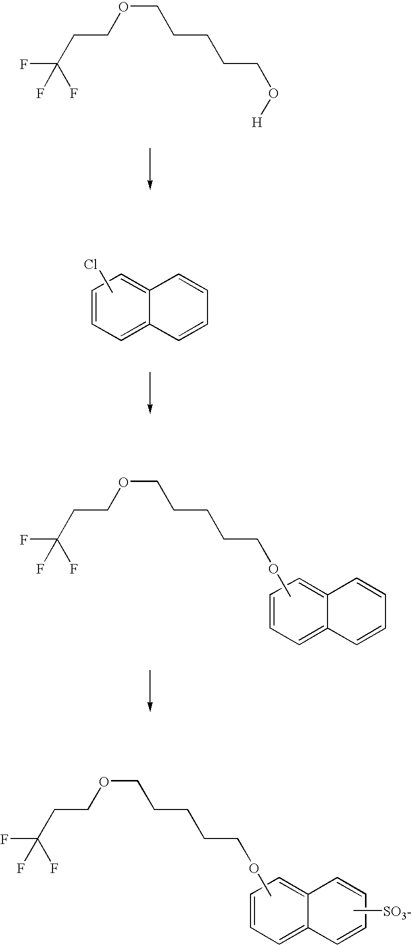

1) The pentane alcohol is obtained analogously to Example 6 by Williamson ether synthesis.

2) Alkoxynaphthalene. The pentanol (0.1 mol), Na—O-t-Bu (0.2 g) and cat. Pd-DBA (5 g) are added to 2-(3-)chloronaphthalene (16 g), the mixture is warmed at 80° C. for 4 hrs, and, after cooling, the alkoxynaphthalene is isolated using conventional laboratory methods.

3) Alkoxynaphthalenesulfonic acid. The alkoxynaphthalene (33 g) is sulfonylated using SO₃ (12 g). For salt formation, the reaction mixture is added to ice/methyl tert-butyl ether, the water phase is extracted with this solvent, and all org. phases are evaporated. Ethanol, then NaOH (4 g) are added, the mixture is warmed briefly and cooled, and the crystals formed are isolated and dried.

### Example 10

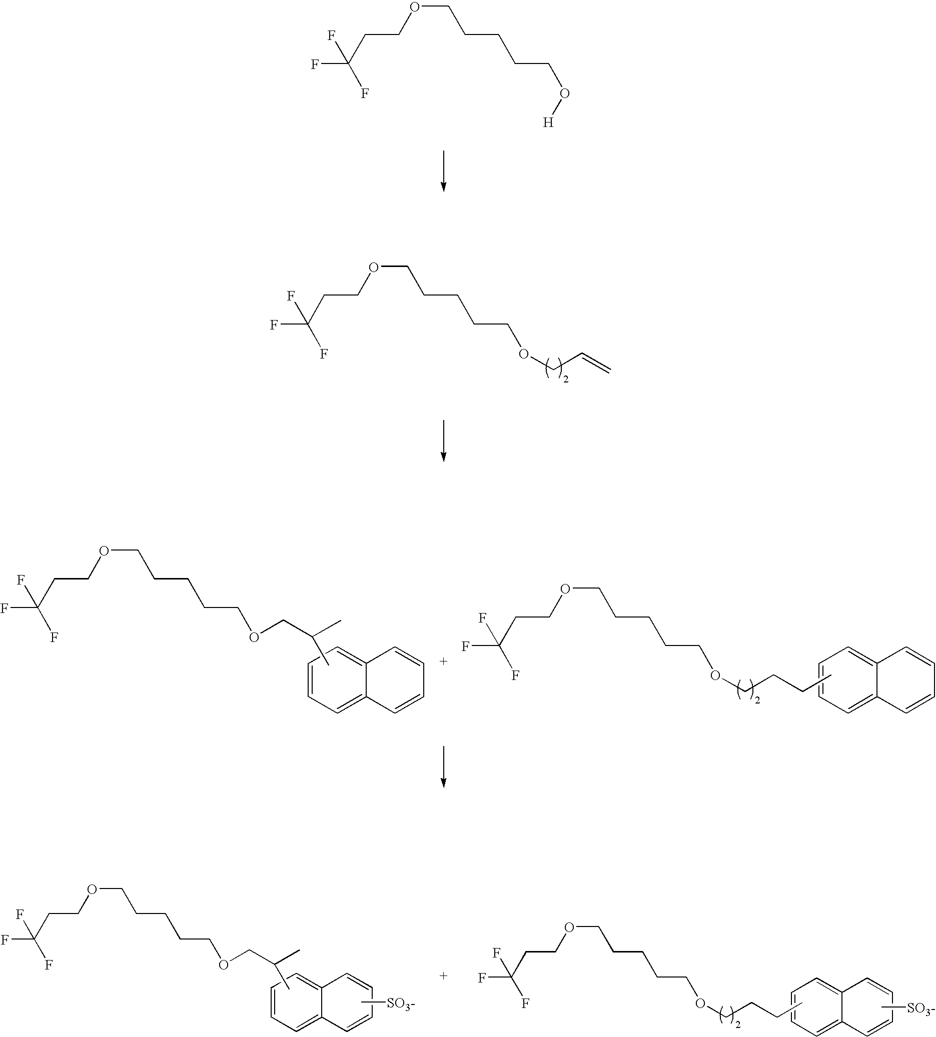

1) The alcohol (20 g), accessible in accordance with Example 6, is stirred at 50° C. for 4 hrs with 1-chlorobut-3-ene (0.1 mol) and Na₂CO₃ (11 g) in THF (100 g), and the homoallyl ether is then isolated using conventional laboratory methods.

2) The homoallyl ether (0.1 mol) is dissolved in chlorobenzene (100 g), naphthalene (13 g) and AlCl₃ (26 g) are added, and the mixture is stirred at 80° C. for 6 hrs, and the alkylnaphthalene is then isolated using conventional laboratory methods.

3) The alkylnaphthalene (0.1 mol) is converted into the alkylnaphthalene-sulfonic acid using sulfur trioxide (12 g). For salt formation, the mixture is added to ice/methyl tert-butyl ether, the water phase is extracted with this solvent, and all org. phases are evaporated. Ethanol, then NaOH (4 g) are added, the mixture is warmed briefly and cooled, and the crystals formed are isolated and dried.

### Example 11

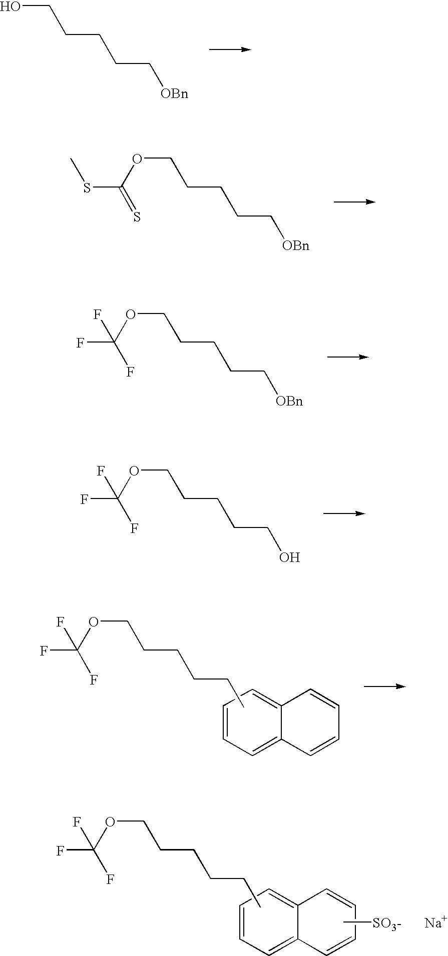

Xanthogenate Variant

1) Xanthogenate. Sodium hydride (0.12 mol; as 60% paraffin suspension) is added to 200 ml of dimethylformamide (DMF), the mixture is cooled to 0° C., and benzyloxypentanol (0.1 mol; dissolved in 30 ml of DMF) is then added. The mixture is stirred for a further 60-90 min. Carbon disulfide (0.2 mol) is then added dropwise at −5-0° C., and the mixture is stirred for a further 2 hrs. Finally, methyl iodide (0.12 mol) is metered in, and the mixture is stirred at 20° C. for 10-14 hrs. The product is isolated and purified using conventional laboratory methods.

2) CF₃ ether. Firstly (HF)₉/Py (160 ml; 65%) and then methyl xanthogenate (100 mmol; in 80 ml of DCM) are added to a suspension of 1,3-dibromo-5,5-dimethylhydantoin (0.29 mol) in DCM (500 ml) at −78° C. The reaction mixture is stirred at −78° C. for a further 1 hr and subsequently warmed to 0° C. over the course of 2 hrs and stirred at this temperature for a further 2 hrs. When the reaction is complete, the product is isolated and purified using conventional laboratory methods.

3) Trifluoromethoxypentanol. The trifluoromethoxypentyl benzyl ether (0.1 mol) is taken up in ethanol (60 g) and hydrogenated at 30° C. with addition of catalyst Pd/C (5%) (0.5 g) and hydrogen (atmospheric pressure). The reaction solution is filtered and evaporated, giving the product. 4) The alcohol (17 g) is dissolved in chlorobenzene (100 g), and naphthalene (13 g) and then AlCl₃ (26 g) are added. After the mixture has been warmed at 100° C. for 12 hrs, it is added to ice, and the organic phase is separated off, filtered and evaporated, and the alkylnaphthalene is recrystallised.

5) The alkylnaphthalene (28 g) is sulfonylated using sulfur trioxide (12 g) without solvent (temp. below 40° C.)/isolation. For salt formation, the mixture is added to ice/methyl tert-butyl ether, the water phase is extracted with this solvent, and all org. phases are evaporated. Ethanol, then NaOH (4 g) are added, the mixture is warmed briefly and cooled, and the crystals formed are isolated and dried.

### Example 12

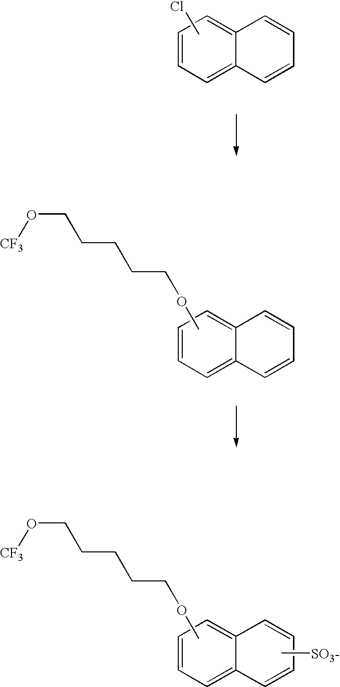

1) The alcohol(17 g), prepared in accordance with Example 11, base Na—O-t-Bu (0.2 g) and cat. Pd-DBA (5 g) are added to 1-(2-)chloronaphthalene (16 g), and the mixture is warmed at 70° C. for 6 hrs, then cooled. Isolation of the product using conventional laboratory methods gives the alkoxynaphthalene.

2) The alkoxynaphthalene (30 g) is sulfonylated using SO₃ (10 g). For salt formation, the mixture is added to ice/methyl tert-butyl ether, the water phase is extracted with this solvent, and all org. phases are evaporated. Ethanol, then NaOH (4 g) are added, the mixture is warmed briefly and cooled, and the crystals formed are isolated and dried,

### Example 13

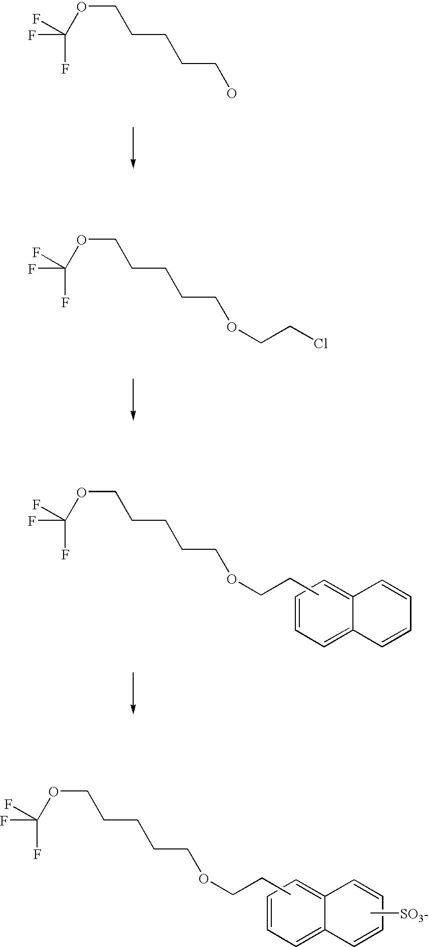

1) The alcohol (17 g), prepared in accordance with Example 11, is etherified at 40° C. using bromochloroethane (14 g) in 200 ml of THF with addition of triethylamine (10 g). The product is isolated using conventional laboratory methods.

2) The chloroethyl ether (23 g) and naphthalene (13 g) are subsequently converted into the alkylnaphthalene at 60° C. in 3 hrs with AlCl₃ catalysis (26 g). The product is likewise isolated using conventional laboratory methods.

3) Alkylnaphthalenesulfonic acid

The alkylnaphthalene (34 g) is sulfonylated using sulfur trioxide (17 g), and, for salt formation, the mixture is added to ice/methyl tent-butyl ether, the water phase is extracted with this solvent, and all organic phases are evaporated. Firstly ethanol, then NaOH (4 g) are added, the mixture is warmed briefly and cooled, and the crystals formed are isolated and dried.

### Example 14

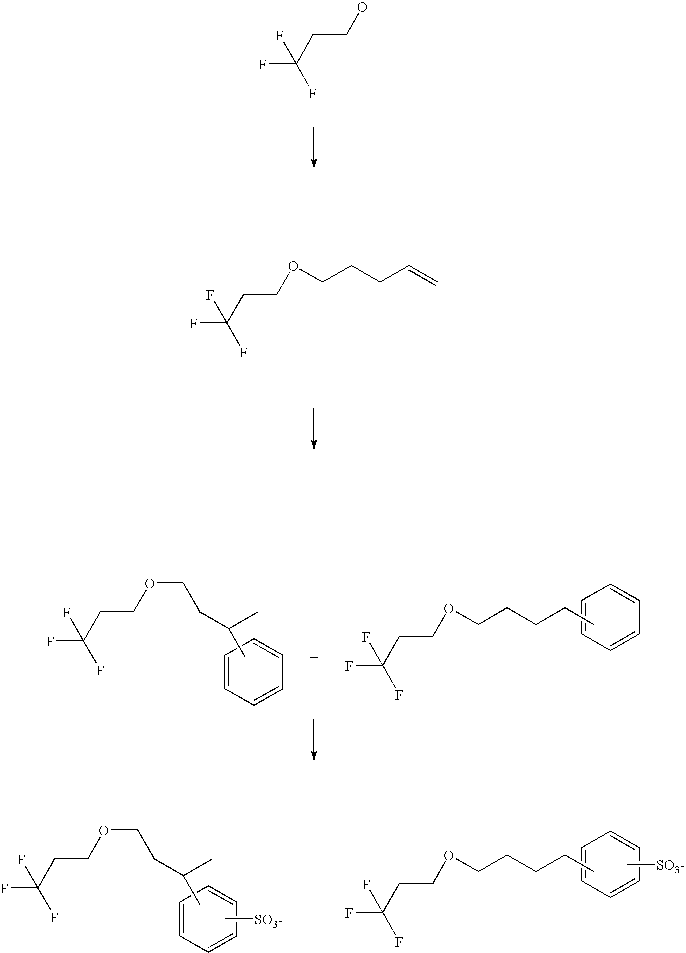

1) 1-Chloropent-4-ene (0.1 mol) and Na₂CO₃ (11 g) in THF (100 g) are added to 3,3,3-trifluoropropan-1-ol (11 g), the mixture is stirred under reflux for 4 hrs, and the allyl ether is then isolated using conventional laboratory methods.

2) 5-(3,3,3-Trifluoropropoxy)pent-1-ene (18 g) is initially introduced in benzene (100 g), then warmed at 60° C. for 6 hrs with AlCl₃ catalysis (26 g) and converted into the corresponding alkylbenzene. The product is isolated by conventional laboratory methods.

3) The alkylbenzene (25 g) is sulfonylated using sulfur trioxide (12 g). For salt formation, the mixture is added to ice/methyl tert-butyl ether, the water phase is extracted with this solvent, and all org. phases are evaporated. Ethanol, then NaOH (4 g) are added, the mixture is warmed briefly and cooled, and the crystals formed are isolated and dried.

### Example 15

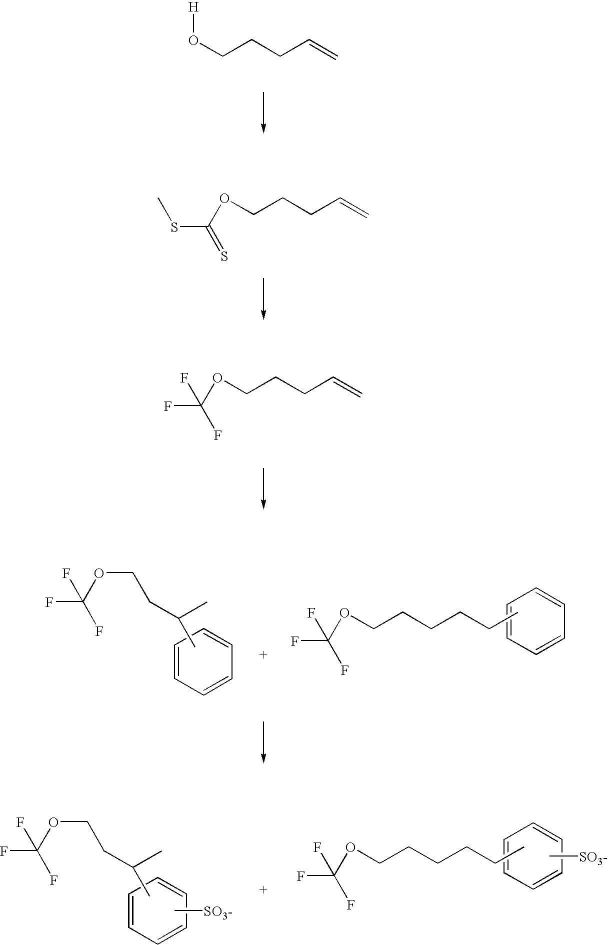

Analogously to Example 11, the intermediate 5-trifluoromethoxypent-1-ene is firstly obtained from pent-4-en-1-ol by means of the xanthogenate method.

1) 5-Trifluoromethoxypent-1-ene (15 g) is initially introduced in benzene (120 g) and converted into the corresponding alkylbenzene with AlCl₃ catalysis (16 g). For isolation, the mixture is quenched using ice, and the organic phase is separated off, filtered and evaporated.

2) The alkylbenzene (22 g) is sulfonylated using sulfur trioxide (17 g). For salt formation, the mixture is added to ice/methyl tert-butyl ether, the water phase is extracted with this solvent, and all org. phases are evaporated. Ethanol, then NaOH (4 g) are added, the mixture is warmed briefly and cooled, and the crystals formed are isolated and dried.

### Example 16

### 3-(Bistrifluoromethylamino)propan-1-ol

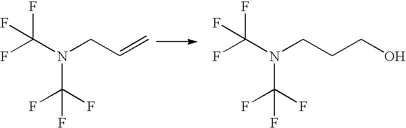

Bistrifluoroallylamine (23.2 g, 120 mmol) is added dropwise at RT to a stirred solution of 9-BBN (240 ml) in THF (200 ml).

After 24 hrs, the reaction mixture is cooled to 0° C., and 3 M NaOH (44 ml, evolution of heat) and 30% H₂O₂ solution (40 ml, countercooled using a dry ice/acetone bath) are subsequently added dropwise. The mixture is heated at 50° C. for 1 hr (the colourless solid redissolves) and then cooled to RT. For work-up, saturated NaCl soln. is added, the phases are separated, the organic phase is again washed by shaking with Na₂SO₃ solution and then dried over sodium sulfate, and the drying agent is filtered off. The solvent is stripped off, and the residue (product) is distilled.

### Example 17

### 1-Bromo-3-(bistrifluoromethylamino)propane

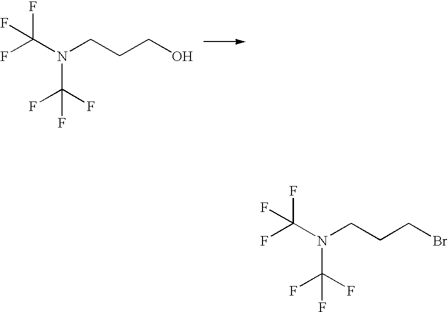

The alcohol (110 mmol) is initially introduced in dry DCM (0.2 molar solution), and triphenylphosphine (154 mmol, 1.4 eq) and subsequently, in portions, tetrabromomethane (CBr₄: 165 mmol, 1.5 eq) are added. The reaction is stirred for 4 hrs and then quenched using sat. NaHCO₃ soln., extracted with MTBE and dried over sodium sulfate.

The crude product formed is isolated by distillation.

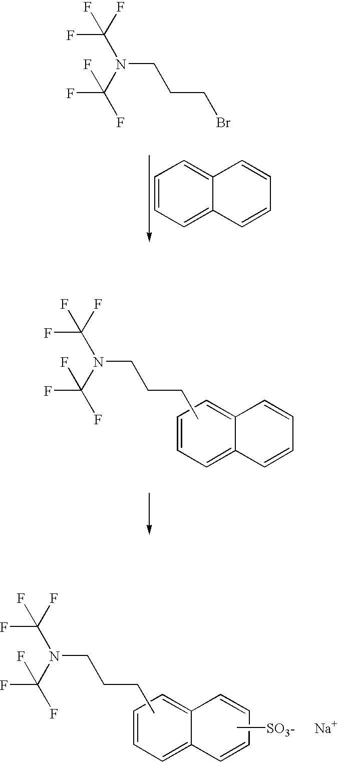

1) 1-Bromo-3-(bistrifluoromethylamino)propane (18.2 g) is dissolved in 100 g of chlorobenzene, and naphthalene (13 g) and then AlCl₃ (13 g) are added. After warming at 100° C. for 12 hrs, the mixture is added to ice, and the organic phase is separated off. The org. phase is evaporated, and the alkylnaphthalene is recrystallised.

2) The alkylnaphthalene (32 g) is sulfonylated using sulfur trioxide (12 g) without solvent (temp. below 40° C.). For salt formation, the mixture is added to ice/methyl tert-butyl ether, the water phase is extracted with this solvent, and all org. phases are evaporated. Ethanol, then NaOH (4 g) are added, the mixture is warmed briefly and cooled, and the crystals formed are isolated and dried.

### Example 18

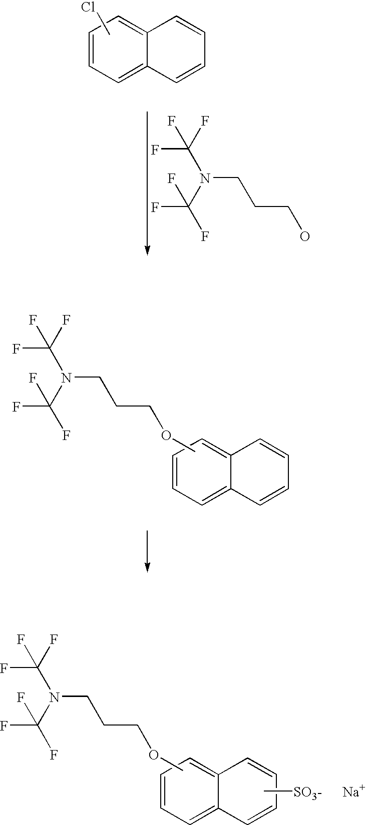

1) 3-(Bistrifluoromethylamino)propan-1-ol (21 g), prepared in accordance with Example 16, and base Na—O-t-Bu (0.2 g) and the catalyst Pd-DBA (5 g) are added to 1-(2-)chloronaphthalene (16 g). After warming (6 hrs; 100° C.) and cooling, the alkoxynaphthalene is isolated using conventional laboratory methods.

2) The alkoxynaphthalene (30 g) is converted into the alkylnaphthalene-sulfonic acid using sulfur trioxide (12 g). For salt formation, the mixture is added to ice/methyl tert-butyl ether, the water phase is extracted with this solvent, and all org. phases are evaporated. Ethanol, then NaOH (4 g) are added, the mixture is warmed briefly and cooled, and the crystals formed are isolated and dried.

### Example 19

### 4-(12-Trifluoromethoxydodecyl)benzenesulfonic acid sodium salt

### (12-Trifluoromethoxydodecyl)benzene

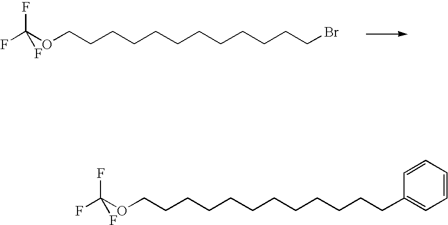

0.5 g of AlCl₃ is added at 0° C. under nitrogen and with stirring to a solution of 3.33 g of 1-bromo-12-trifluoromethoxydodecane in 10 ml of benzene, and the mixture is stirred at 20-25° C. for 48 hrs (analogously to Gilman; Meals; J. Org. Chem., 1943; 126). In order to isolate the product, the reaction mixture is added to ice, the organic phase is separated off and filtered, and the solvent is removed at 50° C. in vacuo, giving a colourless, wax-like solid.

### 4-(12-Trifluoromethoxydodecyl)benzenesulfonic acid sodium salt

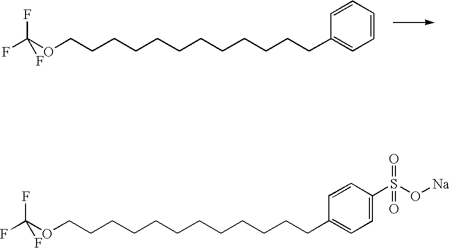

2.67 g of chlorosulfonic acid are added at 0° C. under nitrogen and with stirring to a solution of 5.8 g of (12-trifluoromethoxydodecyl)benzene in 26 ml of 1,2-dichloroethane, and the mixture is stirred at 15-20° C. for 3 hrs. In order to isolate the product, the reaction mixture is added to ice, the organic phase is separated off and filtered, and the solvent is removed at 40° C. in vacua. 18 ml of 1 M NaOH are added to the residue, the mixture is stirred, and the water is then carefully removed from the aqueous phase in vacuo, giving a pale-yellow, amorphous solid.

H-NMR (DMSO; 300 MHz): 1.24-1.28 (10H, m), 1.30 (2H, m), 1.52 (2H, m), 1.58 (2H, m), 1.68-1.70 (4H, m), 2.63 (2H, t), 3.94 (2H, t), 7.17 (2H, m), 7.53 (2H, m)

MS: 392 (M⁺-Na⁺)

### Example 20

### 4-(12-Trifluoromethylsulfanyldodecyl)benzenesulfonic acid sodium salt

### (12-Trifluoromethylsulfanyldodecyl)benzene

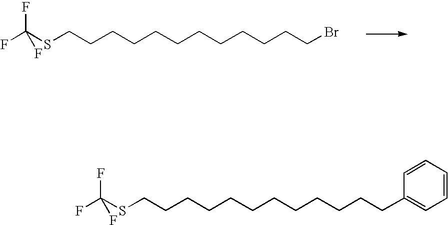

Analogously to the preparation of (12-trifluoromethoxydodecyl)benzene, 0.5 g of AlCl₃ is metered at 0° C. under nitrogen and with stirring into a solution of 3.49 g of 1-bromo-12-trifluoromethylsulfanyldodecane in 10 ml of benzene, and the mixture is stirred at 20-25° C. for 48 hrs (analogously to Gilman; Meals; J. Org. Chem., 1943; 126). In order to isolate the product, the reaction mixture is added to ice, the organic phase is separated off and filtered, and the solvent is removed at 50° C. in vacuo, giving a virtually colourless solid.

### 4-(12-Trifluoromethylsulfanyldodecyl)benzenesulfonic acid sodium salt

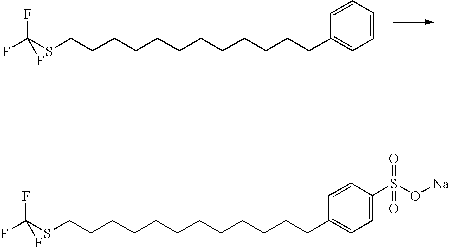

2.43 g of chlorosulfonic acid are added at 0° C. under nitrogen and with stirring to a solution of 5.6 g of (12-trifluoromethylsulfanyldodecyl)benzene in 24 ml of 1,2-dichloroethane, and the mixture is stirred at 15-20° C. for 3 hrs. In order to isolate the product, the reaction mixture is added to ice, the organic phase is separated off and filtered, and the solvent is removed at 40° C. in vacuo. 16 ml of 1 M NaOH are added to the residue, the mixture is stirred, and the water is then carefully removed from the aqueous phase in vacua, giving an amorphous solid.

H-NMR (DMSO; 300 MHz): 1.22-1.27 (8H, m), 1.31-1.34 (4H, m), 1.52 (2H, m), 1.58 (2H, m), 1.64-1.68 (4H,), 2.63 (2H, t), 2.84 (2H, t), 7.17 (2H, m), 7.53 (2H, m)

MS: 409 (M⁺-Na⁺)

### Example 21

### 4[10-(Bistrifluoromethylamino)decyl]benzenesulfonic acid sodium salt

### (10-Phenyldecyl)bistrifluoromethylamine

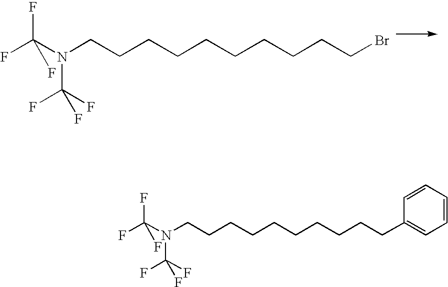

Analogously to the preparation of (12-trifluoromethoxydodecyl)benzene, 0.5 g of AlCl₃ is metered at 0° C. under nitrogen and with stirring into a solution of 3.72 g of (10-bromodecyl)bistrifluoromethylamine in 10 ml of benzene, and the mixture is stirred at 20-25° C. for 48 hrs (analogously to Gilman; Meals; J. Org. Chem., 1943; 126). In order to isolate the product, the reaction mixture is added to ice, the organic phase is separated off and filtered, and the solvent is removed at 50° C. in vacua, giving an amorphous solid.

### 4[10-(Bistrifluoromethylamino)decyl]benzenesulfonic acid sodium salt

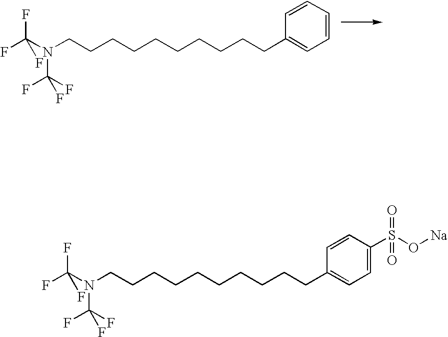

2.89 g of chlorosulfonic acid are added at 0° C. under nitrogen and with stirring to a solution of 7.00 g of (10-phenyldecyl)bistrifluoromethylamine in 29 ml of 1,2-dichloroethane, and the mixture is stirred at 15-20° C. for 3 hrs. In order to isolate the product, the reaction mixture is added to ice, the organic phase is separated off and filtered, and the solvent is removed at 40° C. in vacuo. 19 ml of 1 M NaOH are added to the residue, the mixture is stirred, and the water is then carefully removed from the aqueous phase in vacua, giving a yellowish, amorphous solid.

H-NMR (DMSO; 300 MHz): 1.23-1.28 (8H, m), 1.52 (2H, m), 1.58 (2H, m), 1.68 (2H, m), 1.73 (2H, m), 2.63 (2H, t), 3.18 (2H, t), 7.17 (2H, m), 7.53 (2H, m)

MS: 409 (M⁺-Na⁺)

### Example 22

### 4-Sulfophenyl 12-(bistrifluoromethylamino)dodecanecarboxylate sodium salt

### 12-(Bistrifluoromethylamino)dodecanecarboxylic acid

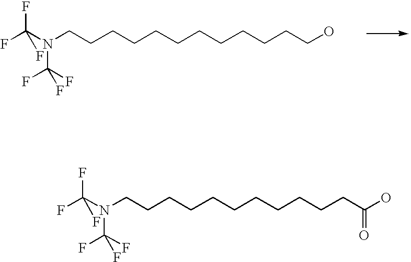

Jones reagent (obtained by addition of 1.68 ml of conc. H₂SO₄ to a solution of 2.56 g of CrO₃ in 11 ml of water at 0° C.) is added at 0° C. under nitrogen and with stirring to a solution of 4.72 g of 12-(bistrifluoromethylamino)-dodecan-1-ol in 56 ml of acetone, and the mixture is stirred at 20° C. for 1 hr. In order to isolate the product, the reaction mixture is filtered, and the solvent is removed from the resultant eluate at 30° C. in vacuo. The crude product is chromatographed over silica gel in a mixture of cyclohexane/ethyl acetate (8:2), giving an amorphous solid.

### 4-Sulfophenyl 12-(bistrifluoromethylamino)dodecanecarboxylate sodium salt

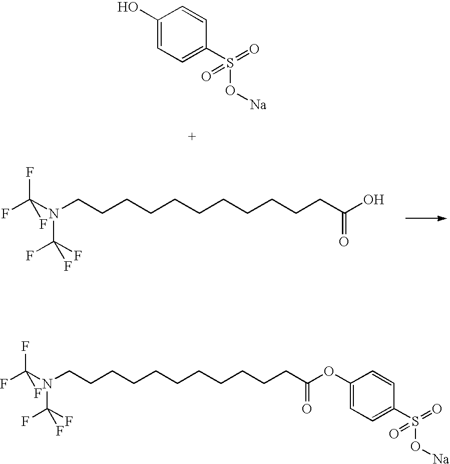

12 g of SOCl₂ are metered at 0° C. under nitrogen and with stirring into a solution of 3.51 g of 12-(bistrifluoromethylamino)dodecanecarboxylic acid in 25 ml of acetone, and the mixture is stirred at 60-70° C. for 0.75 hr, and the excess SOCl₂ and about 50% of the toluene are then removed by distillation. 10 ml of THF, 2.45 g of 4-hydroxyphenylsulfonic acid sodium salt and 4 ml of triethylamine are then added successively at 0-10° C. to the acid chloride remaining in toluene, and the mixture is stirred at 25° C. for 4 hours. In order to isolate the product, the reaction mixture is filtered, and the solvents are removed from the resultant eluate at 30-40° C. in vacuo, giving a partially crystalline solid.

H-NMR (DMSO; 300 MHz): 1.23-1.31 (14H, m), 1.55 (2H, m), 1.73 (2H, m), 2.50 (2H, m), 3.18 (2H, t), 4.11 (2H, t), 7.25 (2H, m), 8.07 (2H, m)

MS: 489 (M⁺-Na⁺)

### Example 23

### 4[12-(Bistrifluoromethylamino)dodecyloxy]benzenesulfonic acid sodium salt

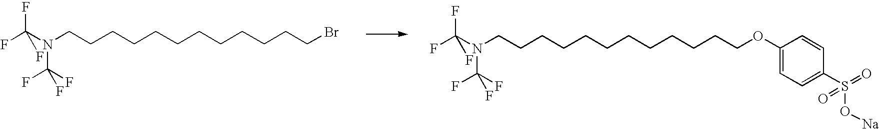

4.84 g of 4-hydroxyphenylsulfonic acid sodium salt and 0.97 g of NaOH (powdered) are added at 20° C. under nitrogen and with stirring to a solution of 8.80 g of (12-bromododecyl)bistrifluoromethylamine in 44 ml of DMF, and the mixture is stirred at 55-65° C. for 24 hrs. In order to isolate the product, the reaction mixture is filtered, and the solvent is removed from the resultant eluate at 40-60° C. in vacuo. 55 ml of ethanol are added to the crude product formed, the mixture is warmed to 40° C., and the suspension is cooled to 0° C. Separation of the solid phase gives a pale-brown solid, which is dried at 30° C. in vacuo.

H-NMR (DMSO; 300 MHz): 1.23-1.28 (14H, m), 1.52 (2H, m), 1.73-1.80 (4H, m), 3.18 (2H, t), 3.98 (2H, t), 6.98 (2H, m), 7.85 (2H, m)

MS: 475 (M⁺-Na⁺)

### Example 24

### Determination of the Biochemical Degradability

The biochemical degradability of the compounds is determined by the Zahn-Wellens test corresponding to the European Commission publication: Classification, Packaging and Labelling of Dangerous Substances in the European Union, Part II—Testing Methods, Annex V—Methods for the Determination of Physico-Chemical Properties, Toxicity and Ecotoxicity, Part B, Biochemical Degradability—Zahn-Wellens Test (0.9.), January 1997, pages 353-357.
Batch volume: 1.5 l
Activated sludge concentration: 1 g of solids/l
Origin of the sludge: treatment plant of Merck KGaA; Darmstadt (not adapted)
Amount of test substances used: about 100 to 200 mg/I as DOC
Aeration: with purified air
Work-up of the samples: filtration (medium-hard filter)
Determination of the DOC: by the difference method using a Dimatec instrument

Further details on the method are given in the above publication and also the OECD Guideline for the testing of chemicals, section 3, degradation and accumulation, method 302 B, page 1-8, adopted: 17.07.92, the contents of which in this respect expressly belong to the disclosure content of the present application.

In addition, besides the degradation of the compound per se in the test, the degradation of the fluorine-containing groups is also observed via a fluoride determination:

Method: ion chromatography

Instrument: Dionex 120

Detector type: conductivity detector

Column: AS9HC

Eluent: sodium carbonate solution, 9 mmol/l

Flow rate: 1 ml/min

Literature: EN ISO 10304-2

### Example 25

### Determination of the Surface Tension

Instrument: Krüss tensiometer (model K12)
Temperature of the measurement solutions: 20° C.
Measurement module employed: ring
Concentration of the measurement solutions: about 0.5 to 3.0 g/l in deionised water

Further details on the method are given in the European Commission publication: Classification, Packaging and Labelling of Dangerous Substances in the European Union, Part II—Testing Methods, Annex V—Methods for the Determination of Physico-Chemical Properties, Toxicity and Ecotoxicity, Part A, Surface Tension (A.5), January 1997, pages 51-57, and also the OECD Guideline for the testing of chemicals, section, physical-chemical properties, method 115, page 1-7, adopted: 27.07.95, the contents of which in this respect expressly belong to the disclosure content of the present application.

1. Compounds containing an arylsulfonate group, a spacer and at least one group Y, where Y stands for CF₃—(CH₂)ₐ—O—, SF₅—, CF₃—(CH₂)ₐ—S—, CF₃CF₂S—, [CF₃—(CH₂)ₐ]₂N— or [CF₃—(CH₂)ₐ]NH—, where a stands for an integer selected from the range from 0 to 5, or 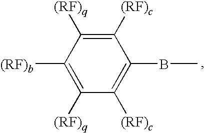
where
Rf stands for CF₃—(CH₂)ᵣ—, CF₃—(CH₂)ᵣ—O—, CF₃—(CH₂)ᵣ—S—, SF₅—(CH₂)ᵣ— or [CF₃—(CH₂)ᵣ]₂N—, [CF₃—(CH₂)ᵣ]NH— or (CF₃)₂N—(CH₂)ᵣ—,
B stands for a single bond, O, NH, NR, CH₂, C(O)—O, C(O), S, CH₂—O, O—C(O), N—C(O), C(O)—N, O—C(O)—N, N—C(O)—N, O—SO₂ or SO₂—O,
R stands for alkyl having 1 to 4 C atoms,
b stands for 0 or 1 and c stands for 0 or 1,
q stands for 0 or 1, where at least one radical from b and q stands for 1, and
r stands for 0, 1, 2, 3, 4 or 5.

2. Compounds according to claim 1, characterised in that the spacer contains no fluorine atoms.

3. Compounds according to claim 1, characterised in that the spacer denotes a saturated or unsaturated, branched or unbranched hydrocarbon unit, where the hydrocarbon unit in the chain or in a branch may optionally be provided with one or more heteroatoms.

4. Compounds according to claim 1, characterised in that they conform to the formula IA or IB 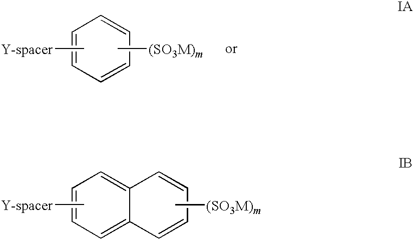
where Y is as defined in claim 1,
spacer is as defined in claim 1
M denotes a metal cation, and
m denotes 1, 2 or 3.

5. Compounds according to claim 1, characterised in that the spacer has one of the following meanings:
—(CH₂)ₙ—,
—(CH₂)—CH(Hal)-(CH₂)₍ₙ₋₁₎—,
—CH═CH—(CH₂)₍ₙ₋₁₎—,
—(CH₂)ₙ—O—,
—(CH₂)ₙ—O—(CH₂)ₚ—,
—CH₂CH═CH—(CH₂)₍ₙ₋₁₎—,
—(CH₂)ₙ₋₁—Ar—(CH₂)₍ₙ₋₁₎—,
—(CH₂)ₙ₋₁—C≡C—(CH₂)ₙ— or
—(CH₂)ₙ-Q-(CH₂)_{n′}—,
where
n and n′, independently of one another, stand for an integer from the range 1 to 30,
Hal denotes Cl, Br or I, and
p stands for an integer from the range 1 to 4,
Ar stands for aryl,
Q stands for O, S or N.

6. Compounds according to claim 1, characterised in that the group Y denotes CF₃—(CH₂)ₐO—, where a=0, 1, 2, 3, 4 or 5, preferably a=0, 1 or 2.

7. Compounds according to claim 1, characterised in that the group Y denotes SF₅.

8. Compounds according to claim 1, characterised in that the group Y denotes CF₃—(CH₂)ₐ—S—, where a=0, 1, 2, 3, 4 or 5, preferably a=0, 1 or 2.

9. Compounds according to claim 1, characterised in that the group Y denotes CF₃—CF₂—S—.

10. Compounds according to claim 1, characterised in that the group Y denotes [CF₃—(CH₂)ₐ]₂N—, where a=0, 1, 2, 3, 4 or 5, preferably a=0, 1 or 2.

11. Compounds according to claim 1, characterised in that the group Y denotes [CF₃—(CH₂)ₐ]NH—, where a=0, 1, 2, 3, 4 or 5, preferably a=0, 1 or 2.

12. Compounds according to claim 1, characterised in that the group Y denotes 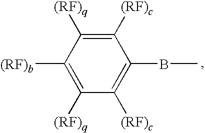
where
Rf stands for CF₃—(CH₂)ᵣ—, CF₃—(CH₂)ᵣ—O—, CF₃—(CH₂)ᵣ—S—, CF₃CF₂—S—, SF₅—(CH₂)ᵣ— or [CF₃—(CH₂)ᵣ]₂N—, [CF₃—(CH₂)ᵣ]NH— or (CF₃)₂N—(CH₂)ᵣ—,
B stands for a single bond, O, NH, NR, CH₂, C(O)—O, C(O), S, CH₂—O, O—C(O), N—C(O), C(O)—N, O—C(O)—N, N—C(O)—N, O—SO₂ or SO₂—O,
R stands for alkyl having 1 to 4 C atoms,
b stands for 0 or 1 and c stands for 0 or 1,
q stands for 0 or 1, where at least one radical from b and q stands for 1, and
r stands for 0, 1, 2, 3, 4 or 5.

13. Process for the preparation of the compounds according to the invention, characterised in that a compound of the formula II
Y-spacer-Z II,
where Y and -spacer- have one of the meanings indicated in claim 1, and
Z denotes OH, Br, Cl or vinyl,
is reacted with the corresponding aromatic compound selected from the group of benzene and naphthalene, and a sulfonation and salt formation are subsequently carried out.

14. Composition comprising at least one compound according to claim 1.

15. Composition according to claim 14, characterised in that a vehicle which is suitable for the respective application and optionally further specific active compounds are present.

16. Composition according to claim 14, characterised in that the composition is a paint or coating preparation, fire-extinguishing composition, lubricant, washing or cleaning composition, de-icer or hydrophobicising agent for textile finishing or glass treatment.

17. Process for the preparation of a composition according to claim 14, characterised in that at least one compound is mixed with a suitable vehicle and optionally with further specific active compounds.

18. A method of achieving an effect comprising using a compound of claim 1 as a surfactant.

19. Use of compounds according to claim 1 as hydrophobicising agents or oleophobicising agents, in particular in the surface modification of textiles, paper, glass, porous building materials or adsorbents.

20. Use of compounds according to claim 1 as antistatic, in particular in the treatment of textiles, such as clothing, carpets and carpeting, upholstery in furniture and automobiles, non-woven textile materials, leather goods, papers and cardboard articles, wood and wood-based materials, mineral substrates, such as stone, cement, concrete, plaster, ceramics, such as glazed and unglazed tiles, earthenware, porcelain, and glasses, and for plastics and metallic substrates.

21. Use of compounds according to claim 1 as additives in preparations for surface coating, such as printing inks, paints, coatings, photographic coatings, special coatings for semiconductor photolithography, such as photoresists, top antireflective coatings, bottom antireflective coatings, or in additive preparations for addition to corresponding preparations.

22. Use of compounds according to one or more of claim 1 as foam stabiliser and/or for supporting film formation, in particular in fire-extinguishing foams.

23. Use of compounds according to claim 1 as interface promoter or emulsifier, in particular for the preparation of fluoropolymers.

24. Use of compounds according to claim 1 as antimicrobial active compound, in particular as reagent for antimicrobial surface modification.
